SEQUENCE LISTING (11 sequences)

SEQ ID NO: 1 - protein, 594 aa, M. crassisigna
MGSSINDEHILSALLQSDDELVGEDSDSEVSDHVSEDDVQSDTEEAFIDEVHEVQPTSSGSEILDEQNVIEQPGSSLASNRTLTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKNFMRNLYMGLTSSFMRKRLEAPTLKRYLRDNISNILPKEVPGTSDDSTEEPVTKKRTYCTYCPSKIRRKANASCKKCKKVICREHNIDMCQSCF

SEQ ID NO: 2 - DNA, 44 bp, Artificial
  Int-3′ITR Gib F
atatgattatctttctagggttaaatcactgaatccgggagcac

SEQ ID NO: 3 - DNA, 40 bp, Artificial
  Int-5′ITR Gib R
cgcagactatctttctagggttaattctatgcccagcacg

SEQ ID NO: 4 - DNA, 40 bp, Artificial
  5′ITR-Int Gib F
cgtgctgggcatagaattaaccctagaaagatagtctgcg

SEQ ID NO: 5 - DNA, 54 bp, Artificial
  5′ITR-3xpA Gib R
attatgatcagaagatctgggatatctataacaagaaaatatatatataataag

SEQ ID NO: 6 - DNA, 43 bp, Artificial
  3xpA-5′ITR Gib F
attttcttgttatagatatcccagatcttctgatcataatcag

SEQ ID NO: 7 - DNA, 40 bp, Artificial
  3′ITR-3xpA Gib F
ataaagtaacaaaacttttaggatcccgagcttggcactg

SEQ ID NO: 8 - DNA, 55 bp, Artificial
  3′ITR-3xpA Gib F
cagtgccaagctcgggatcctaaaagttttgttactttatagaagaaattttgag

SEQ ID NO: 9 - DNA, 46 bp, Artificial
  3′ITR-Int Gib R
tcccggattcagtgatttaaccctagaaagataatcatattgtgac

SEQ ID NO: 10 - DNA, 41 bp, Artificial
  PolyA-Transpose Gib F
tgtgccaatcttgcttctgagaattcaccccaccagtgcag

SEQ ID NO: 11 - DNA, 40 bp, Artificial
  CMVTO2-Transpose Gib R
tcgttgatagacgaacccatggtggcggcctttgccaaag

(19) United States
(12) Patent Application Publication
(10) Pub. No.: US 2022/0177854 A1
(43) Pub. Date: Jun. 9, 2022

(54) ADENO-ASSOCIATED VIRAL VECTOR PRODUCER CELL LINES

(71) Applicant: GlaxoSmithKline Intellectual Property Development Limited, Brentford Middlesex (GB)

(72) Inventors: Simon Alexander CHANAS, Stevenage (GB); Conrad VINK, Stevenage (GB)

(21) Appl. No.: 17/284,848

(22) PCT Filed: Oct. 15, 2019
(86) PCT No.: PCT/EP2019/077879

§ 371 Date: Apr. 13, 2021

(30) Foreign Application Priority Data
Oct. 17, 2018 (GB) 1816919.3

(51) Int. Cl. C12N 7/00; C12N 15/86; C12N 9/12

(52) U.S. Cl. CPC C12N 7/00; C12N 15/86; C12N 9/1241; C12N 2750/14162; C12N 2800/30; C12N 2710/10043; C12N 2750/14143; C12N 2820/002

(57) ABSTRACT

The invention relates to an adeno-associated virus (AAV) vector producer cell comprising nucleic acid sequences encoding AAV rep and cap genes, helper virus genes, and a DNA genome of the AAV vector; the AAV rep gene comprising an intron, the intron comprising a transcription termination 5 sequence with a first recombination site located upstream and a second recombination site located downstream of the transcription termination sequence; and the nucleic acid sequences all integrated together at a single locus within the AAV vector producer cell genome. The invention also relates to methods for producing the AAV vector producer cell lines.

### CROSS REFERENCE TO RELATED APPLICATIONS

This application is a § 371 of International Application No. PCT/EP2019/077879, filed 15 Oct. 2019, which claims the benefit of GB Application No. 1816919.3, filed 17 Oct. 2018.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on 11 Mar. 2021, is named “PB66657 US Sequence Listing” and is 8 Kb in size.

### FIELD OF THE INVENTION

The invention relates to AAV vector producer cell lines, methods for producing the same and nucleic acid vectors for use in said methods.

### BACKGROUND TO THE INVENTION

Adeno-associated virus (AAV) was discovered in 1965, as a contaminant of adenovirus preparations. AAV has a linear single-stranded DNA (ssDNA) genome of approximately 4.7-kilobases (kb), with two 145 nucleotide-long inverted terminal repeats (ITR) at the termini. The ITRs flank two open reading frames (rep and cap genes) encoding a series of Rep (replication) and Cap (capsid) polypeptides. The Rep polypeptides (Rep78, Rep68, Rep62 and Rep40) are non-structural proteins, which are involved in replication, rescue and integration of the AAV genome. The Cap proteins, (VP1, VP2 and VP3) are structural proteins, which form the virion capsid. AAV has been classified as a Dependoparvovirus (a genus in the Parvoviridae family) because it requires co-infection with helper viruses such as adenovirus, herpes simplex virus (HSV) or vaccinia virus for productive infection in cell culture. For example, the adenovirus provides genes that are required to be expressed for AAV replication and virion production: E1A, E1B, E2A, E4 and the VA (Atchison et al. (1965) Science 149:754; Buller et al. (1981) J. Virol. 40:241).

AAV vectors have demonstrated transduction and long-term gene expression, and have the ability to infect both dividing and quiescent cells. Furthermore, AAV is not currently known to cause disease and, therefore, causes little to no toxicity and inflammation in vivo. These characteristics have led to AAV becoming a desirable vector for gene therapy applications.

Several methods of AAV vector production in cells lines are commonly used, and can be divided into two differing strategies. The first strategy is based on the wild-type helper virus-free transient co-transfection of all elements (plasmid expressing AAV vector DNA (transgene flanked by AAV ITRs), plasmid expressing rep and cap genes, plasmid expressing helper virus genes, commonly isolated from adenovirus), which are required for AAV vector production in host cells, such as HEK293 cells (Xia et al. (1996) J. Virol. 70:8098). Although the transient co-transfection method generates high titres of AAV vectors that are free of adenovirus, the process is very labour-intensive, expensive and difficult to scale up for large scale production.

The second strategy involves wild-type helper virus (e.g. wild-type adenovirus) infection of cell lines that stably harbour the rep and cap genes, as well as the transgene flanked by the AAV ITRs. Although the wild-type adenovirus inducible method can be scaled up in cultures and produce AAV vectors with high titres, it is very challenging to completely remove the adenovirus from the AAV product. Contamination of wild-type adenovirus is highly undesirable in view of vector safety and specificity.

The disadvantages of current methods of AAV vector production may be overcome by providing a stable producer cell line for large-scale clinical grade production of recombinant AAV vectors for clinical use. However, creation of a cell line constitutively expressing the rep and cap genes has been difficult due to the cytotoxic and antiproliferative effects of the Rep proteins on the host cell that could severely limit its usefulness as an AAV vector producer cell line. For example, Rep78 has been shown to induce p53 independent apoptosis, attributable in part to the DNA binding and ATPase-helicase activities of Rep78. Furthermore, Rep78 is known to inhibit cell cycle progression, in particular, including complete arrest within S phase. Rep78, together with Rep68, also produces nicks in the cellular chromatin, inducing a DNA damage response leading to G₁ and G₂ blocks. In addition, Rep78 has been shown to affect cAMP signal transduction pathways, which play a central role in regulating cell growth and development (Schmidt et al. (2000) J. Virol. 74:9441; Berthet et al. (2005) PNAS 102:13634; Schmidt et al. (2002) J. Virol. 76:1033).

As such, stable cell lines constitutively expressing Rep proteins are not able to survive to reach the cell density required to produce AAV vectors in a large-scale bioreactor. Therefore, it would be desirable to have a stable producer cell line having stably integrated in its genome all the genetic elements and control systems required for inducible production of a recombinant AAV vector to overcome one or more disadvantages associated with existing methods and cell lines.

### SUMMARY OF THE INVENTION

The present inventors have developed a novel AAV vector producer cell line wherein all of the nucleic acid sequences encoding the viral genes (AAV and helper virus) and transgene essential for recombinant AAV production are integrated together at a single locus within the AAV vector producer cell genome and in which expression of the Rep proteins may be regulated to allow efficient manipulation of the producer cell line, for example during cell line generation, cell banking and cell expansion. The expression of Rep is controlled by using an expression control system in which all the rep transcripts from both the rep promoters, P5 and P19, are prematurely terminated until a time point wherein expression of Rep is desired. The advantage of this system is that it is possible to maintain all the native promoters of the rep and cap genes in order to maintain the correct stoichiometry of the various rep and cap gene transcripts required for efficient AAV vector production. Furthermore, as the expression control system is contained in an intron within the rep gene, which is spliced out during RNA processing, it also has the added advantage that the integrity of the mRNA of the rep and cap genes are not affected.

For producing the novel AAV vector producer cell line of the invention, the inventors have developed a new way of making producer cell lines which involves the use of nucleic acid vectors comprising a non-mammalian origin of replication and the ability to hold at least 25 kilobases (kb) of DNA, such as bacterial artificial chromosomes, carrying all the adeno-associated virus (AAV) and helper genes, and the transgene essential for recombinant AAV vector production.

The use of a nucleic acid vector comprising a non-mammalian origin of replication and which has the ability to hold at least 25 kb of DNA (i.e. large-construct DNA) has several advantages. In the first instance, the vectors can first be manipulated in non-mammalian cells (e.g. microbial cells, such as bacterial cells) rather than mammalian host cells, making them much easier to work with (e.g. bacterial artificial chromosomes can first be manipulated in E. coli). Once the nucleic acid vector has been prepared, it can be introduced into a mammalian host cell and any cells into which the nucleic acid vector has integrated into one or several of the endogenous chromosomes can be selected for in order to isolate a stable cell line.

Introduction of the nucleic acid vector into mammalian host cells also occurs in a single step, helping to reduce selection pressure and silencing timeframe. This allows for faster screening of potential producer cells and reduces the cost of materials because only a single vector is used, as compared to previous methods which involve screening for each of the multiple plasmid vectors. In particular, use of this system reduces the cost of plasmid manufacture, reduces requirement for transfection reagents (e.g. Polyethylenimine (PEI)), reduces the amount of Benzonase™ treatment required (there is a reduced amount of DNA in the viral harvest, therefore less Benzonase™ is needed to remove the excess in downstream processing) and reduces costs of testing (there is no need to test for residual plasmid in the viral product). These advantages are particularly pertinent to large-scale industrial production of recombinant AAV vectors for therapeutic application, which must adhere to GMP requirements.

Furthermore, because all the nucleic acid sequences encoding all the elements essential for recombinant AAV vector production are cloned contiguously within the same nucleic acid vector, when the vector is introduced into mammalian host cells, all of the genes incorporated in the vector will integrate at one locus within the endogenous mammalian host cell genome. This makes it easier to select for stable clones in which none of the required genes for AAV production have integrated into a region of the genome that can cause gene silencing. This might occur to one or more genes when the genes required for AAV vector production are provided on several plasmids which can integrate randomly at different loci within the host cell genome.

Thus, the present invention provides an AAV vector producer cell that is simple and optimised for large-scale industrial production for therapeutic applications and overcomes the disadvantages associated with existing cell lines. Furthermore, the invention provides methods for producing said AAV vector producer cell and nucleic acid vectors for use therein.

Therefore, according to a first aspect of the invention, there is provided an adeno-associated virus (AAV) vector producer cell comprising nucleic acid sequences encoding:

AAV rep and cap genes,

helper virus genes, and

a DNA genome of the AAV vector;

wherein the AAV rep gene comprises an intron, said intron comprising a transcription termination sequence with a first recombination site located upstream and a second recombination site located downstream of said transcription termination sequence; and

wherein said nucleic acid sequences are all integrated together at a single locus within the AAV vector producer cell genome.

According to a further aspect of the invention, there is provided a nucleic acid vector comprising a non-mammalian origin of replication and the ability to hold at least 25 kilobases (kb) of DNA, characterized in that said nucleic acid vector comprises nucleic acid sequences encoding:

AAV rep and cap genes;

helper virus genes; and

a DNA genome of an AAV vector;

wherein the rep gene comprises an intron, said intron comprising a transcription termination sequence with a first recombination site located upstream and a second recombination site located downstream of the transcription termination sequence; and

wherein the nucleic acid sequences encoding the AAV rep and cap genes, each of the helper virus genes and the DNA genome of the AAV vector are arranged as individual expression cassettes within the nucleic acid vector.

According to yet a further aspect of the invention, there is provided a method of producing a stable AAV vector producer cell line, comprising:

(a) introducing the nucleic acid vector as defined herein into a culture of mammalian host cells; and

(b) selecting within the culture for a mammalian host cell which has the nucleic acid sequences encoded on the vector integrated into an endogenous chromosome of the mammalian host cell.

In a further aspect of the invention, there is provided an AAV vector producer cell obtained by the method described herein.

In a further aspect of the invention, there is provided a method of producing a replication defective AAV vector, comprising:

(a) introducing the nucleic acid vector as defined herein into a culture of mammalian host cells; and

(b) selecting within the culture for a mammalian host cell which has the nucleic acid sequences encoded on the vector integrated into an endogenous chromosome of the mammalian host cell; and

(c) further culturing the selected mammalian host cell under conditions in which the replication defective AAV vector is produced.

In yet a further aspect of the invention, there is provided a replication defective AAV vector obtained by the method described herein.

### DESCRIPTION OF DRAWINGS/FIGURES

FIG. 1: A Peggy Sue western blot showing expression of Rep proteins for Rep2Cap2.

FIG. 2: A Peggy Sue western blot showing expression of Rep proteins for Rep2Cap5.

FIG. 3: A bar chart showing % GFP positive CHO cells transduced by recombinant AAV vectors produced using transient transfection methods with helper virus genes under conditional transcription.

FIG. 4: A bar chart showing % GFP positive CHO cells transduced by recombinant AAV vectors produced using transient transfection methods with Cre dependent rep/cap expression plasmid.

FIG. 5: A schematic diagram showing a nucleic acid vector according to one embodiment of the invention.

### DETAILED DESCRIPTION OF THE INVENTION

### Definitions

Unless defined otherwise, all technical and scientific terms used herein have the same meaning as is commonly understood by one of skill in the art to which this invention belongs. All patents and publications referred to herein are incorporated by reference in their entirety.

The term “comprising” encompasses “including” or “consisting” e.g. a composition “comprising” X may consist exclusively of X or may include something additional e.g. X+Y.

The term “consisting essentially of” limits the scope of the feature to the specified materials or steps and those that do not materially affect the basic characteristic(s) of the claimed feature.

The term “consisting of” excludes the presence of any additional component(s).

The term “about” in relation to a numerical value x means, for example, x±10%, 5%, 2% or 1%.

The terms “transfection”, “transformation” and “transduction” as used herein, may be used to describe the insertion of the non-mammalian or viral vector into a target cell. Insertion of a vector is usually called transformation for bacterial cells and transfection for eukaryotic cells, although insertion of a viral vector may also be called transduction. The skilled person will be aware of the different non-viral transfection methods commonly used, which include, but are not limited to, the use of physical methods (e.g. electroporation, cell squeezing, sonoporation, optical transfection, protoplast fusion, impalefection, magnetofection, gene gun or particle bombardment), chemical reagents (e.g. calcium phosphate, highly branched organic compounds or cationic polymers) or cationic lipids (e.g. lipofection). Many transfection methods require the contact of solutions of vector DNA to the cells, which are then grown and selected for a marker gene expression.

Inserted genetic materials exist in cells either stably or transiently. For stable transfection, the inserted genetic material is integrated into the host cell genome and sustain transgene expression even after host cells have replicated. Therefore, the term “stably transfected” or “stable cell” refers to cell lines which are able to pass introduced genetic material to their progeny (i.e. daughter cells), either because the transfected DNA has been incorporated into the endogenous chromosomes or via stable inheritance of exogenous chromosomes.

In contrast to stably transfected genes, transiently transfected genes are only expressed for a limited period of time and are not integrated into the host cell genome. Transiently transfected genetic materials may be lost by environmental factors and cell division.

The term “producer cell” refers to a cell line with AAV packaging genes (rep and cap genes), the helper virus genes as required and a DNA genome of the recombinant AAV vector, (e.g. a transgene of interest flanked by the two AAV inverted terminal repeats (ITRs)), stably integrated into the host cell genome. It will be understood by a person skilled in the art that the nucleic acid vectors described herein may be used to generate the producer cell lines. It will be further understood that the producer cells described herein do not refer to cells in which the natural AAV provirus has been integrated.

The term “gene” is a well-known term in the art. As used herein, a gene includes an expressed nucleic acid sequence that encodes a protein or is transcribed into a functional RNA product. Generally, a gene includes the expressed nucleic acid sequence, with operably linked regulatory sequences including, but not limited to, promoters, enhancers, operators and terminators. Two sequences are “operably linked” if they are arranged in cis to act in an expected manner in relationship to each other. The terms “expressed”, “expression” mean the overall process by which the information encoded in a nucleic acid, typically a gene, is converted into ribonucleic acid and/or a protein or a post-translationally modified version thereof.

A “transgene” as used herein is a nucleic acid sequence encoding a gene of interest, such as, without limitation, a gene to allow for genetic or drug selection (e.g. a gene conferring antibiotic resistance, or a reporter gene). Alternatively, the gene may be a therapeutic gene which replaces or augments the function of a defective gene, used for immunisation against agents to provoke an immunogenic response.

Some methods of recombinant AAV vector manufacture known in the art involves transient transfection of a transfer vector (nucleic acid vector comprising a transgene of interest flanked by the two AAV ITRs) into a cell line stably expressing the packaging genes, and in some cases additionally the helper virus genes. This process reduces the disadvantages of an entirely transient transfection process but does not completely remove them. Thus, such a hybrid method of recombinant AAV vector manufacture arguably has the combined complexity of both stable and transient approaches.

Producer cell-based AAV vector manufacture requires a more complex cell line development phase than that a process utilising transient transfection. However, this process has the advantages of requiring fewer starting materials and operations during manufacture, such that there are fewer elements that can go wrong. Moreover, the simplified manufacturing process as disclosed herein is better for scalability to large-scale, industrially applicable production system and is better able to meet the demand of large patient populations.

The terms “viral vector” or a “virion” in the context of AAV, refers to an AAV particle (i.e. AAV vector, also referred to elsewhere in the patent application as “recombinant AAV vector”) suitable for carrying genetic material to be transferred into a host cell. The AAV vector may be referred to as empty or full, that is to say, does not contain or contains a DNA genome of the AAV vector, respectively. In the case of AAV vectors, the AAV genome has been modified to remove the rep and cap genes from between the two AAV ITR sequences. The DNA genome of the AAV vector of the invention (i.e. AAV vector) typically comprises a transgene flanked by the two AAV ITRs. It will be understood that the term “nucleic acid vector” does not refer to or include “viral vectors” (for example, AAV vector encapsidating a DNA genome for transfer into a host cell). Rather, the term “nucleic acid vector” refers to a genetic construct.

The term “intron” or “intron sequence” refers to a non-coding sequence within a gene that is removed by RNA splicing during modification of the precursor messenger RNA into mature messenger RNA (mRNA). Thus, the term refers to both the DNA sequence within a gene and the corresponding sequence in the unprocessed precursor messenger RNA transcript. Where a nucleic acid sequence encoding a gene comprises nucleic acids which together with an inserted sequence form a consensus splice donor/acceptor sequence, an intron may be inserted at this position. The inserted sequences are then spliced out during post-transcriptional processing. Methods for insertion of nucleic acid sequences encoding an intron into an expressed sequence are well known in the art and any such methods may be used to do so.

Alternatively, the use of an intron downstream of the enhancer/promoter region and upstream of the cDNA insert has been shown to increase the level of gene expression. The increase in expression depends on the particular cDNA insert.

Accordingly, the nucleic acid vector of the present invention may include introns such as human chorionic gonadotrophin intron, human beta globin intron, rabbit beta globin intron II or a chimeric human beta globin-immunoglobulin intron. In one embodiment, the intron is a human beta globin intron and/or a rabbit beta globin intron II.

The term “transcription termination sequence” “transcription terminator” refers to a nucleic acid sequence that mediates transcriptional termination by providing signals in the newly synthesised RNA transcript that trigger processes which release the transcript RNA from the transcriptional complex (i.e. RNA polymerase). In eukaryotic transcription, the transcription termination sequence is a polyadenylation (polyA) signal sequence, which enables host factors to add a polyadenosine (polyA) tail to the end of the nascent mRNA during transcription. The polyA tail is a stretch of up to 300 adenosine ribonucleotides which protects mRNA from enzymatic degradation and also aids in translation. Accordingly, the nucleic acid vectors of the present invention may include a polyA signal sequence such as the simian virus 40 (SV40) early or late polyA signals, the human beta globin or rabbit beta globin polyA signals, the human insulin polyA signal, or the bovine growth hormone polyA signal. In one embodiment, the polyA signal sequence is simian virus 40 (SV40) polyA signal. In another embodiment, the polyA signal sequence is the human beta globin polyA signal.

The terms “recombination site” and “recombinase” are well known in the art and are used to refer to components of the process of site-specific recombination. For example, members of the tyrosine recombinases, namely Cre and FLP, have been effectively employed in the art as molecular tools for use in eukaryotes to mediate site-specific DNA insertions or targeted DNA deletions. The Cre recombinase recombines a pair of short target sequences, or recombination sites, called LoxP sequence. Similarly, the FLP recombinase recognises and targets the FRT sequence. By way of further example, recombinase includes transposase, which recombines a pair of short target sequences, or recombination sites, known as transposon inverted terminal repeat (transposon ITR). DNA transposons, also known as class 2 transposable elements, are flanked at both ends by terminal inverted repeats. The inverted repeats are complements of each other (the repeat at one end is a mirror image of, and composed of complementary nucleotides to, the repeat at the opposing end).

The term “locus” as used in the art refers to a specific location on a chromosome, or any region of genomic DNA that is considered to be a discrete genetic unit for the purpose of formal linkage analysis or molecular genetic studies. For the purposes of integration of nucleic acid sequences encoding the genes essential for production of a recombinant AAV vector in a host cell genome as in the present invention, the discrete genetic unit is two DNA base pairs on the endogenous host cell genome in between which the sequences are inserted (e.g. insertion site). Accordingly, the term “locus” as used herein, does not refer to a large region of genomic DNA, for example a megabase-size region containing a large gene family, but a specific location on the genome.

The term “nucleic acid vector” refers to a vehicle which is able to artificially carry foreign (i.e. exogenous) genetic material into another cell, where it can be replicated and/or expressed. Examples of vectors include non-mammalian nucleic acid vectors, such as bacterial artificial chromosomes (BACs), yeast artificial chromosomes (YACs), P1-derived artificial chromosomes (PACs), cosmids or fosmids. The term “nucleic acid vector DNA” refers to the DNA of the nucleic acid vector, comprising the nucleic acid sequences encoding various genes or elements therein.

The term “non-mammalian origin of replication” refers to a nucleic acid sequence where replication is initiated, and which is derived from a non-mammalian source. This enables the nucleic acid vectors described herein to stably replicate and segregate alongside endogenous chromosomes in a suitable host cell (e.g. a microbial cell, such as a bacterial or yeast cell) so that it is transmittable to host cell progeny, except when the host cell is a mammalian host cell. In mammalian host cells, nucleic acid vectors with non-mammalian origins of replication will either integrate into the endogenous chromosomes of the mammalian host cell or be lost upon mammalian host cell replication. For example, nucleic acid vectors with non-mammalian origins of replication such as bacterial artificial chromosomes (BAC), P1-derived artificial chromosome (PAC), cosmids or fosmids, are able to stably replicate and segregate alongside endogenous chromosomes in bacterial cells (such as E coli). However, if they are introduced into mammalian host cells, the BAC, PAC, cosmid, fosmid or plasmids will either integrate or be lost upon mammalian host cell replication. Yeast artificial chromosomes (YAC) are able to stably replicate and segregate alongside endogenous chromosomes in yeast cells. However, if they are introduced into mammalian host cells, the YAC will either integrate or be lost upon mammalian host cell replication. Therefore, in this context, the nucleic acid vectors described herein act as reservoirs of DNA (i.e. for the genes essential for AAV vector production) which can be easily transferred into mammalian cells to generate stable producer cell lines for recombinant AAV vector production. Examples of non-mammalian origins of replication include bacterial origins of replications, such as oriC, oriV or oriS, or yeast origins of replication, also known as Autonomously Replicating Sequences (ARS elements).

In one embodiment, the nucleic acid vector comprises a non-mammalian origin of replication and is able to hold at least 25 kilobases (kb) of DNA. In one embodiment, the nucleic acid vector has the ability to hold at least 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340 or 350 kb of DNA. It will be understood that references to “ability to hold” has its usual meaning and implies that the upper limit for the size of insert for the nucleic acid vector is not less than the claimed size (i.e. not less than 25 kb of DNA).

The term “endogenous chromosomes” or “endogenous genome” refers to genomic DNA found in the host cell prior to generation or introduction of an exogenous nucleic acid vector, such as the nucleic acid vector described herein. Preferably, the nucleic acid vector is a bacterial artificial chromosome.

The term “promoter” refers to a sequence that drives gene expression. In order to drive a high level of expression, it may be beneficial to use a high efficiency promoter. Examples of suitable promoters may include a promoter such as the human cytomegalovirus (CMV) immediate early promoter, spleen focus-forming virus (SFFV) promoter, Rous sarcoma virus (RSV) promoter, or human elongation factor 1-alpha (pEF) promoter. In one embodiment, the promoter is an inducible promoter (also referred to elsewhere in the application as a conditional promoter) to allow for temporal regulation of the expression of a gene to which it is linked. Inducible promoters and inducible expression systems are well known in the art.

The term “selectable marker” refers to a gene that will help select cells actively expressing a nucleic acid sequence. Examples of suitable selection markers include, enzymes encoding resistance to an antibiotic (i.e. an antibiotic resistance gene), e.g., kanamycin, neomycin, puromycin, hygromycin, blasticidin, or zeocin. Another example of suitable selection markers are fluorescent proteins, for example green fluorescent protein (GFP), red fluorescent protein (RFP) or blue fluorescent protein (BFP).

“Gene amplification” refers to a process by which specific DNA sequences of the genome (i.e. genes) are disproportionately replicated in relation to the other sequences in the genome such that the amplified DNA sequences become present in a higher copy number than was initially present in the genome before such disproportionate replication. “Amplified” or “amplification” as used herein with reference to a gene or nucleic acid sequence refers to a gene or nucleic acid sequence present in two or more copies in a host cell line by virtue of gene amplification.

References to an “amplifiable selection marker gene” as used herein refers to a gene which permits the amplification of that gene under appropriate growth conditions. The amplifiable selection marker gene is capable of responding either to an inhibitor or lack of an essential metabolite by amplification to increase the expression product (i.e. the expression of the protein encoded by the amplifiable selection marker gene). In one embodiment, the amplifiable selection marker gene may be characterized as being able to complement an auxotrophic host.

The term “expression construct” or “expression cassette” as used herein refers to a functional expression unit, capable of driving the expression of one or more incorporated polynucleotides, that is to say a DNA sequence containing one or more genes and sequences that control their expression. Expression cassettes usually include the polynucleotide and the components necessary for the transcription and translation of the polynucleotide. For example, the cassette may include a nucleic acid sequence (i.e. recombinant DNA) including a promoter, a translational initiation signal, a transcriptional terminator (e.g. a polyA signal sequence) and/or a self-cleaving peptide sequence (e.g. P2A sequence). In one embodiment, the individual expression cassette comprises a promoter and/or a transcriptional terminator. In one embodiment, the individual expression cassette comprises two genes separated by an IRES that are both transcribed from a single promoter. For the avoidance of doubt, the rep and cap genes, which produce several transcripts from 3 different promoters that are then spliced into 7 different proteins, form a single contiguous genetic element and cannot be separated from each other due to the compact nature of the AAV genome. As such, the skilled person will understand that the rep and cap genes are comprised in a single expression cassette. Therefore, expression cassettes may comprise more than one promoter.

In one embodiment, all of the expressions cassettes in the nucleic acid vector are arranged so that they transcribe in the same direction. This has previously been shown to improve overall expression of the expression cassettes in a construct (Throm et al., (2009) Blood 113: 5104-5110).

### Adeno Associated Virus (AAV) Vector Producer Cell

According to one aspect of the invention, there is provided an adeno-associated virus (AAV) vector producer cell comprising nucleic acid sequences encoding:

AAV rep and cap genes,

helper virus genes, and

a DNA genome of the AAV vector;

wherein the AAV rep gene comprises an intron, said intron comprising a transcription termination sequence with a first recombination site located upstream and a second recombination site located downstream of said transcription termination sequence; and

wherein said nucleic acid sequences are all integrated together at a single locus within the AAV vector producer cell genome.

It will be understood that these nucleic acid sequences encoding the various genes are present as individual expression cassettes which prevents any risk of recombination to form replication competent viruses. For avoidance of doubt, the nucleic acid sequences encoding the rep and cap genes are present in the same (i.e. one) expression cassette.

In one embodiment, the AAV vector producer cell is a mammalian cell. In a further embodiment, the mammalian cell is selected from a HEK293 cell, CHO cell, Jurkat cell, K562 cell, PerC6 cell, HeLa cell or a derivative or functional equivalent thereof. In yet a further embodiment, the mammalian host cell is a HEK293 cell, or derived from a HEK293 cell. Such cells could be adherent cell lines (i.e. they grow in a single layer attached to a surface) or suspension adapted/non-adherent cell lines (i.e. they grow in suspension in a culture medium). In a further embodiment, the HEK293 cell is a HEK293T cell.

The term “HEK293 cell” refers to the Human Embryonic Kidney cells that were transfected with fragments of mechanically sheared adenovirus 5 (Ad5) DNA (Graham et al. (1977) J. Gen. Virol. 36:59). The early region 1 (E1) of the adenovirus 5 genome, consisting of the transcription units E1A and E1B, is stably integrated into the HEK293 cell genome. Since HEK293 cells stably express Ad5 E1A and E1B, production of recombinant AAV in HEK293 producer cells only requires transfection with the remaining essential adenovirus helper genes (E2A, E4 and VA) and the AAV genome. As such, HEK293 is commonly used in AAV production. Other examples of suitable commercially available cell lines include T REX™ (Life Technologies) cell lines. Accordingly, in one embodiment, the helper virus genes comprise all or part of helper virus genes E2A, E4 and VA.

### Adeno-Associated Virus

Adeno-associated viruses (AAV) is part of the genus Dependoparvovirus, which belongs to the family Parvoviridae. AAV is a small, non-enveloped, icosahedral virus with single-stranded DNA (ssDNA) genome of approximately 4.7 kilobases (kb) to 6 kb in length. Several serotypes have been discovered, with AAV serotype 2 (AAV2) as the most extensively examined serotype so far.

The AAV genome consists of two open reading frames, rep and cap genes (also referred to elsewhere in the application as rep/cap gene), flanked by two 145 base inverted terminal repeats (ITRs). These ITRs base pair to allow for synthesis of the complementary DNA strand. The rep and cap genes are translated to produce multiple distinct proteins: the rep gene encodes the proteins Rep78, Rep68, Rep52, Rep40, which are required for the AAV life cycle; the cap gene encodes VP1, VP2, VP3, which are the capsid proteins. When constructing an AAV transfer plasmid, the transgene is placed between the two ITRs, and rep and cap genes are supplied in trans. This is to ensure that the recombinant AAV vector produced by the host cell is replication defective.

The AAV rep coding sequences encode at least those replication proteins that are necessary for viral genome replication and packaging into new virions. The rep gene will generally encode at least one large Rep protein (i.e. Rep78/68) and one small Rep protein (i.e. Rep52/40), however in the embodiments described herein, the rep gene does not need to encode all of the AAV Rep proteins. Therefore, in one embodiment, the Rep proteins comprise the Rep78 protein and the Rep52 and/or Rep40 proteins. In an alternative embodiment, the Rep proteins comprise the Rep68 and the Rep52 and/or Rep40 proteins. In a further embodiment, the Rep proteins comprise the Rep68 and Rep52 proteins, Rep68 and Rep40 proteins, Rep78 and Rep52 proteins, or Rep78 and Rep40 proteins. In a yet further embodiment, the Rep proteins comprise the Rep78, Rep68, Rep52 and Rep40 proteins.

The AAV cap gene encodes the structural proteins that form a functional AAV capsid (i.e. can package DNA and infect target cells). Typically, the cap gene will encode all of the AAV capsid subunits, but less than all of the capsid subunits may be encoded as long as a functional capsid is produced. In one embodiment, the Cap proteins comprise VP1, VP2 and/or VP3.

The AAV ITR sequences comprise 145 bases each and are the only cis-acting elements necessary for AAV genome replication and packaging into the capsid. Typically, the ITRs will be at the 5′ and 3′ ends of the vector genome and flank the heterologous nucleic acid (transgene) but need not be contiguous thereto. The ITRs can be the same or different from each other.

An AAV ITR may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, bovine AAV, goat AAV, shrimp AAV, or any other AAV now known or later discovered. An AAV ITR need not have the native terminal repeat sequence (e.g. a native AAV ITR sequence may be altered by insertion, deletion, truncation and/or missense mutations), as long as the terminal repeat mediates the desired functions, e.g., replication, virus packaging, and/or integration, and the like.

References to AAV as used herein, includes, but is not limited to, AAV serotype 1 (AAV1), AAV serotype 2 (AAV2), AAV serotype 3 (including serotypes 3A and 3B) (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 12 (AAV12), AAV serotype 13 (AAV13), snake AAV, avian AAV, bovine AAV, canine AAV, equine AAV, ovine AAV, goat AAV, shrimp AAV, and any other AAV now known or later discovered. See, e.g. Fields et al. Virology, volume 2, chapter 69 (4th ed., Lippincott-Raven Publishers).

References to AAV may include artificial AAV serotypes which include, without limitation, AAV with a non-naturally occurring capsid protein. Such an artificial capsid may be generated by any suitable technique, using one AAV serotype sequence (e.g. a fragment of a VP1 capsid protein) in combination with heterologous sequences which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanised” AAV capsid.

In one embodiment, the nucleic acid sequences encoding the rep and cap genes and/or the DNA genome of the AAV vector (i.e. the AAV nucleic acid sequences) are derived from AAV1, AAV2, AAV3, AAV4, AAV5, AAV6, AAV7, AAV8, AAV9, AAV10, AAV11, AAV12, AAV13 or combinations thereof. In a further embodiment, the nucleic acid sequences encoding the rep and cap genes and/or the DNA genome of the AAV vector are derived from AAV2, AAV5, AAV8 and/or AAV9.

Alternatively, in one embodiment the rep gene sequences are from an AAV serotype which differs from that which is providing the cap sequences. Therefore, in one embodiment, the rep sequences are fused in frame to cap sequences of a different AAV serotype to form a chimeric AAV vector. For example, in one embodiment, the rep gene is derived from AAV2 and the cap gene is derived from AAV2 or AAV5 to produce AAV2-like and AAV5-like particles, respectively. These may be named rep2cap2 and rep2cap5.

The genomic sequences of various serotypes of AAV, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching AAV nucleic acid and amino acid sequences.

Tissue specificity is thought to be determined by the capsid serotype and, therefore, pseudotyping of AAV vectors can be used to alter their tropism range. This makes AAV a useful system for preferentially transducing specific cell types. Without being bound by theory, Table 1 summaries the optimal serotypes for transduction of specific tissues:

TABLE 1
Optimal AAV serotype(s) for transduction of a given organ

| Tissue | Optimal Serotype |
|---|---|
| CNS | AAV1, AAV2, AAV4, |
| | AAV5, AAV8, AAV9 |
| Heart | AAV1, AAV8, AAV9 |
| Kidney | AAV2 |
| Liver | AAV2, AAV3, AAV5, |
| | AAV7, AAV8, AAV9, |
| | AAV10 |
| Lung | AAV4, AAV5, AAV6, AAV9 |
| Pancreas | AAV8 |
| Photoreceptor Cells | AAV2, AAV5, AAV8 |
| RPE (Retinal Pigment | AAV1, AAV2, AAV4, AAV5, AAV8 |
| Epithelium) | |
| Skeletal Muscle | AAV1, AAV6, AAV7, AAV8, AAV9 |
| Brain | AAV4, AAV9, AAV10 |

References to “pseudotyping” refer to the mixing of a capsid and genome from different viral serotypes. These serotypes are denoted using a slash, for example, AAV2/5 indicates a virus containing the genome of AAV serotype 2 packaged in the capsid from AAV serotype 5. Use of these pseudotyped viruses can improve transduction efficiency, as well as alter tropism. For example, AAV2/5 targets neurons that are not efficiently transduced by AAV2/2, and is distributed more widely in the brain, indicating improved transduction efficiency. Many of these hybrid viruses have been well characterized in the art.

### Helper Virus Genes

In addition to rep and cap genes, AAV requires a helper virus or plasmid containing genes necessary for AAV replication because AAV does not have the ability to replicate on its own. In the absence of helper viruses, AAVs may incorporate into the host cell genome, at a specific site of chromosome 19. Helper virus sequences necessary for AAV replication are known in the art, for example see Cell & Gene Therapy Insights, “Gene Therapy and Viral Vectors: Advances and Challenges” (Cell Gene Therapy Insights 2016; 2 (5), 553-575). Typically, these sequences will be provided by a helper adenovirus or herpesvirus vector. The helper virus genes encode proteins and non-coding RNA.

In one embodiment, the helper virus genes are derived from adenovirus. In a further embodiment, the adenovirus is selected from adenovirus 2 and adenovirus 5. In one embodiment, the helper virus genes comprise E1A, E1B, E2A, E4 and the VA genes.

Some of the helper genes may be expressed by the mammalian host cell line while other helper genes are introduced by a vector. For example, HEK 293 cells (ATCC CRL-1573) constitutively produce adenoviral E1A and E1B proteins. Thus, for production of recombinant AAV, only the helper genes required for the production of a recombinant AAV vector, such as E2A, E4 and VA, are introduced into the HEK293 host cell.

In one embodiment, the helper virus genes comprise all or part of each of E4, E2A and VA genes derived from adenovirus, in particular adenovirus 2. It has been found that not all of the native adenovirus genes are required for AAV replication, for example only the E4 34 kD protein encoded by open reading frame 6 (ORF 6) of the E4 gene is required for AAV replication. Therefore, in a further embodiment, the helper virus genes comprise an E4 ORF6 coding region, an adenovirus E2A 72 kD coding region (coding for the E2A 72 kD DNA-binding protein) and a VA gene. In a yet further embodiment, the helper virus genes additionally comprise adenovirus E1A and E1B genes.

In an alternative embodiment, the helper virus genes are derived from herpesvirus. In a further embodiment, the herpesvirus is selected from: herpes simplex virus (HSV), Epstein-Barr Virus (EBV), cytomegalovirus (CMV) and pseudorabies virus (PRV).

Each of the helper virus genes may be controlled by the respective original promoter or by heterologous promoters.

It has been reported that the Adenovirus helper genes that are required for AAV production in HEK 293 cells (E2A, E4 and VA) are potentially toxic to host cells (Ferrari et al., 1996, Journal of Virology 70: 3227-3234). When transfected into mammalian cells, the native promoters of these helper genes are constitutively active. Accordingly, in one embodiment, the one or more of the helper virus genes are under transcriptional control. In one embodiment, all of the helper virus genes are under transcriptional control. In yet another embodiment, E4, E2A and VA helper virus genes are under transcriptional control. In a further embodiment a CMV-TO2 promoter is operably linked to E2A gene and/or E4 gene. In yet a further embodiment, TetO operator sequence is operably linked to a native promoter of VA.

By integrating the helper virus genes required for AAV vector production into the host cell genome, it will be understood that this method may be considered a helper virus-free method because it does not require co-infection with a wild-type helper virus. This therefore avoids contamination of wild-type helper virus (e.g. adenovirus) which is highly undesirable in view of vector safety and specificity.

### Rep Repression

A major difficulty in generating a producer cell line in which all the genetic elements required for a recombinant AAV vector are stably integrated into the host cell genome is the constitutive expression of Rep proteins, which are well known to be cytotoxic (Yang et al., 1994, J. Virol. 68:4847-4856) and cytostatic (Schmidt et al., 2000, J. Virol. 74:9441-9450). This means that cells stably expressing the Rep protein will not survive to reach the density required to produce recombinant AAV vectors in a large-scale bioreactor. This difficulty is compounded when generating a HEK293 based producer cell line, owing to E1A-mediated activation of rep gene promoters, p5 and p19. A further layer of complexity in regulating Rep expression is the location of the p19 promoter, which is situated within the coding region of the Rep proteins expressed by p5 promoter (Rep78 and Rep68). As a result, manipulation of the p19 promoter will inevitably cause mutations in coding sequences of Rep78 and Rep68, which is likely to result in the disruption of the structure and functions of these essential Rep proteins. Furthermore, disruption to the native rep promoters will affect the correct stoichiometry of the various Rep and Cap proteins required for efficient AAV vector production.

Therefore, Rep expression needs to be tightly regulated during producer cell growth and highly induced during AAV vector production.

The AAV vector producer cell of the present invention comprises a dual splicing switch within the rep gene for controlling expression of Rep proteins. The dual splicing switch is an intron comprising an excisable transcription termination sequence (“terminating intron”). The excisable termination sequence is a termination sequence flanked by a pair of recombination sites. There may be additional nucleic acid sequences within the flanking recombination sites in addition to the transcription termination sequence. Introns and examples of intron sequences are well known in the art. In one embodiment, the intron is an intron from the human chorionic gonadotrophin gene.

The terminating intron is positioned within the Rep coding region. The Rep coding region is modified such that when inserted, the 5′ and 3′ ends of the intron and the nucleic acid sequence of the Rep coding region with which they are contiguous, respectively, form a splice doner/acceptor sequence, to enable splicing out of the terminating intron during RNA processing. For example, on the exon side immediately 5′ of the intron there may be an NC A G sequence and immediately 3′ of the intron is a G nucleotide. Thus, the intron may be inserted into an AAG \hat{ } G (where \hat{ } denotes an insertion site) sequence in the rep gene to provide the final sequence is AAG/GTPuAGU-middle of intron-CAG/G (Pu denoting a purine). Therefore, the termination intron may be inserted anywhere in the rep gene there is a AAGG or CAGG sequence.

In one embodiment, the terminating intron is positioned within Rep coding region downstream of the p19 promoter. In this way, the terminating intron is positioned within a reading frame shared by all four Rep proteins, such that expression of all four rep gene products can be simultaneously controlled.

When the terminating intron is active, transcription of the rep gene is prematurely terminated at the transcription termination sequence, such that production of full length rep transcripts is prevented and Rep expression is inhibited. In this way, levels of Rep production are sufficiently low or completely absent to alleviate the toxicity to the cells and permit propagation of the cells bearing the inactivated rep gene during cell growth.

The transcription termination sequence may be any sequence that is capable of transcription termination. In one embodiment, the transcription termination sequence is the polyadenylation (polyA) signal sequence. In a further embodiment, the terminating intron comprises one or more polyA signal sequences in tandem, such as 2, 3, 4, 5, 6, 7, 8, 9, 10 or more polyA signal sequences in tandem. In a preferred embodiment, the termination intron comprises 3 polyA signal sequences in tandem.

It has been shown by Qiao et al (2002 J. Virol. 76:13015-13027) that a combination of a gene and one or more polyA signal sequences have a synergistic effect in transcription termination. Therefore, in one embodiment, the transcription termination sequence may be a gene. In a further embodiment, the transcription termination sequence is a combination of a polyA signal sequence and a gene. In yet a further embodiment, the termination sequence is two or more polyA signal sequences in tandem, such as 2, 3, 4 5, 6, 7, 8, 9 or more, followed by a gene. In one embodiment, the termination sequence is three polyA signal sequences in tandem followed by a gene. For convenience of establishing a cell line with the rep gene under control of the dual splicing switch, in one embodiment, the gene of the transcription terminating sequence is a selectable marker, such as, without limitation, hygromycin, puromycin or blasticidin S resistance genes.

The rep gene is activated in the presence of a recombinase enzyme (recombinase) as the terminating intron is removed from the rep gene. The recombinase splices out the intron through recombination events, or inverts, the region between the two recombination sites containing the termination sequence. Inverting the termination sequence also has the effect of stopping transcriptional inhibition. In this way, expression of the full length of the rep gene is restored. The remainder of the intron is precisely removed from the full-length precursor mRNA via RNA splicing, restoring the coding sequence of the rep gene to produce the four Rep proteins. This control of Rep expression is referred to as a “dual splicing switch” because two splicing events occur (DNA and RNA splicing) before the transcribed sequence is capable of being translated into the Rep proteins.

The intron remnant in the rep gene after removal of the transcription termination sequence may be long enough in size to act as a stuffier to mitigate formation of replication competent AAV in the event that the rep/cap gene inserts between the ITRs of the transfer vector. For example, in one exemplary embodiment, the remnant intron increases the rep/cap gene size by 404 bp, which is enough to make it too large for the AAV capsid packaging limit.

Site-specific DNA recombinases are widely used in multicellular organisms to manipulate the structure of the genomes and, in turn, control gene expression. These enzymes, derived from bacteria and fungi, catalyse directionally sensitive recombination reactions between short target site (i.e. recombination site) sequences that are specific to each recombinase. Many types of site-specific recombination systems are known in the art, and any suitable recombination system may be used in the present invention. For example, in one embodiment the recombination site(s) are selected or derived from the int/att system of lambda phage, the Cre/lox system of bacteriophage P1, the FLP/FRT system of yeast, the Gin/gix recombinase system of phage Mu, the Cin recombinase system, the Pin recombinase system of E. coli and the R/RS system of the pSR1 plasmid, or any combination thereof. The most widely used recombinases are Cre and FLP, which recognise LoxP and FRT recombination sites, respectively. In one embodiment, the recombinase is a Cre recombinase or an FLP recombinase. In one embodiment, the Cre recombinase is a codon optimised Cre recombinase. In one embodiment, the recombination site is a LoxP site. In one embodiment, the recombination site is an FRT site.

Transposon/transposase systems are well known in the art (Pray, L. (2008) Transposons: The jumping genes. Nature Education 1 (1):204). In one embodiment, the recombination site(s) and recombinase is a transposon/transposase system. Type 1 transposons remain in place and replicate themselves for insertion at other locations rather than the desired “cut and paste” mechanism of type 2 transposons, wherein segments of DNA move from one place to another. Therefore, in one embodiment, the transposon is a type 2 transposon. In one embodiment, the transposon is not a type 1 transposon. In one embodiment, the recombination site(s) is a transposon inverted terminal repeat (transposon ITR). In one embodiment, the recombinase is a transposase. In one embodiment, the transposon ITR and transposase is eukaryotic. In embodiment, the recombination site and recombinase is a eukaryotic transposon/transposase system. In one embodiment, the transposon ITR and transposase are from the same species. Suitable transposon ITRs include but are not limited to, Sleeping Beauty, Tc1-like transposon from Rana pipiens, piggyBac transposon from Trcihoplusia ni (T. ni), hAT-like transposon Tol2 from Oryzias latipes, and transposons from Macdunnoghia crassisigna (M. crassisigna), Bactrocera minuta, Eumeta japonica, or Helicoverpa armigera. In one embodiment, the transposon ITR and transposase are from M. crassisigna.

It has been reported that it is possible, through mutation of 3 amino acids (R372A, K375A, D450N) in the cabbage looper moth (Trichoplusia ni) transposase used in the piggyBac system to create an excision+integration−phenotype (Li et al., 2013 “PiggyBac transposase tools for genome engineering” PNAS 110: E2279-E2287, the mutated trans). In this way expression of the transposase by addition of DOX to cells stably transfected with such a construct would result in an irreversible removal of the transcriptional terminators downstream of the rep promoters, resulting in greater Rep expression.

The transposase from Macdunnoughia crassisigna is 98.82% identical to that from Trichoplusia ni. Yusa et al. (Yusa K et al “A hyperactive piggyBac transposase for mammalian applications, 2011, PNAS 108: 1531-1536) found 7 amino acid substitutions (I30V, S103P, G165S, M282V, S509G, N538K, N571S) in the Trichoplusia ni transposase that resulted in a hyperactive phonotype. These substitutions were applied to the M. crassisigna transposase amino acid sequence. Additionally, 3 amino acid substitutions found by Li et al. (2013, PNAS 110: E2279-E2287) to result in an excision+integration−phonotype in the Trichoplusia ni transposase (R372A, K375A, D450N) were also applied to the M. crassisigna transposase sequence.

The modified M. crassisigna transposase amino acid sequence (SEQ ID NO: 1). In one embodiment, the amino acid sequence encoding the transposase comprises the amino acid sequence of SEQ ID NO: 1. In one embodiment, the amino acid sequence encoding transposase from Trichoplusia ni comprises the mutations R372A, K375A and D450N. In one embodiment, the transposase comprises the mutations R372A, K375A and D450N. In one embodiment, the amino acid sequence encoding transposase from M. crassisigna comprises the mutations I30V, S103P, G165S, M282V, S509G, N538K, N571S. In one embodiment, the transposase comprises the mutations I30V, S103P, G165S, M282V, S509G, N538K, N571S. In one embodiment, the amino acid sequence encoding transposase from M. crassisigna comprises the mutations I30V, S103P, G165S, M282V, S509G, N538K, N571S, R372A, K375A and D450N. In one embodiment, the transposase comprises the mutations I30V, S103P, G165S, M282V, S509G, N538K, N571S, R372A, K375A and D450N.

A number of methods may be used to introduce the recombinase into the producer cell stably expressing the rep gene comprising the termination intron. The recombinase may be provided to the AAV vector producer cell in protein form or as a nucleic acid sequence encoding a recombinase gene. Any methods for introducing a foreign protein or nucleic acid sequence encoding a protein of interest into a cell are well known in the art may be used to introduce the recombinase into the AAV vector producer cell. In one method, recombinase enzymes may be provided in the medium for transport across the cell membrane, for example by lipofection. In another method, a nucleic acid sequence encoding a recombinase may be transferred into the producer cell. Any gene transfer method well known in the art may be applicable. Accordingly, in one embodiment, the AAV vector producer cell further comprises nucleic acid encoding a recombinase gene. However, addition of a gene transfer step in large-scale AAV vector production for therapeutic use may be undesirable, from a safety aspect (viral-mediated gene transfer) or cost aspect (non-viral mediated gene transfer).

Therefore, a separate recombinase gene transfer step may be avoided by producing an AAV vector producer cell by stably transfecting the recombinase gene into the producer cell genome. In one embodiment, the AAV vector producer cell genome comprises a nucleic acid sequence encoding a recombinase gene. In a further embodiment, the nucleic acid sequence encoding the recombinase gene is integrated together with the nucleic acid sequences encoding the AAV rep and cap genes, the helper virus genes and the DNA genome of the AAV vector at a single locus within the AAV vector producer cell genome.

Where an AAV vector producer cell has a recombinase gene stably integrated into its genome, expression of the recombinase gene will need to be repressed until a time when induction of the rep gene is desired. Therefore, a recombinase control system is required. In the absence of a recombinase control system, the recombinase gene will be constitutively expressed to produce recombinase enzymes, which will in turn recognise the recombination sites and splice out the region there between containing the termination sequence resulting production of the Rep proteins. Therefore, in one embodiment the AAV vector producer cell further comprises a recombinase control system.

The recombinase control system is any system capable of sequestering the recombinase enzyme. The recombinase control system may act to control expression of the recombinase gene, to control translation of the recombinase gene transcript, or control the recombinase enzyme activity.

In one embodiment, the recombinase control system comprises a recombinase gene under the control of an inducible promoter (i.e. conditional promoter), explained further below.

In a further embodiment, the recombinase control system comprises a mutated steroid hormone receptor ligand-binding domain (LBD) operably linked to the recombinase gene. Certain recombinases have a propensity to translocate into the nucleus of mammalian cell. For example, it has been shown that Cre protein contains certain determinant sequences that allow for active transport into the nucleus (Andreas et al., 2002, Nucleic Acids Res 27:4703-4709). In order to gain control of recombinase activity at the protein level, chimeric recombinases fused to steroid hormone receptor ligand-binding domains (LBD) have been created. The LBD of such chimeric recombinases are able to interact with synthetic agonists, but incapable of binding to physiologic steroids. In the absence of a synthetic agonist, the binding domain interacts with the heat shock protein complex present in the cytoplasm, resulting in impaired recombinase translocation into the nucleus and decreased activity because of steric hindrance. Conversely, in the presence of a synthetic agonist, the ligand unbound domain does not interact with the cytoplasmic proteins and recombination is free to occur.

In one embodiment the LBD is a estrogen receptor ligand binding domain. In this case, the synthetic agonist is tamoxifen. In a further embodiment, the estrogen receptor ligand binding domain is ERT2. ERT2 is a estrogen receptor ligand binding domain with a higher affinity for tamoxifen. In one embodiment, the recombinase, optionally a codon optimised recombinase, is flanked by ERT2. Casanova et al. (2002, Genesis 34:208-214) generated a tamoxifen-inducible fusion protein generated by fusing two ERT2 domains onto both ends of a codon improved Cre recombinase for recombination studies in the brain. The fusion protein was reported to be cytoplasmic in the absence of tamoxifen and translocated into the nucleus upon tamoxifen administration. In the absence of tamoxifen, no background recombinase activity was detected.

In a further embodiment, the recombinase control system comprises a recombinase gene under the control of an inducible promoter and a steroid hormone receptor ligand-binding domain operably linked to the recombinase gene.

### Inducible Promoters

Inducible expression system is advantageous in applications in which it is desirable to provide regulation over expression of specific nucleic acid sequence(s). In the present invention, exogenous control over recombinase regulated Rep expression is obtained by operably linking an inducible promoter to the nucleic acid sequences encoding recombinase gene. An inducible promoter in the context of this invention comprises an associated response element. In a further embodiment, exogenous control over expression of the helper virus genes is also obtained by operably a transcriptional control element to one or more of the helper virus genes. The transcription control element may be an inducible promoter or simply a response element, where a native promoter is to be used. For example, only the TetO operator sequences may be used with the native promoter of the helper gene, which is a Pol III promoter and would not transcribe correctly is a standard Pol II inducible promoter is used.

In one embodiment, the inducible promoter is a Tet responsive promoter (Ptet promoter). The Tet responsive promoter comprises at least one Tet operon. A Tet operon (Tetracycline-Controlled Transcriptional Activation) may be used in a method of inducible gene expression, wherein transcription is reversibly turned on or off in the presence of the antibiotic tetracycline or one of its derivatives (e.g. doxycycline (DOX)). In one embodiment, the inducing agent is tetracycline or a derivative thereof.

In nature, the Ptet promoter expresses TetR, the repressor, and TetA, the protein that pumps tetracycline antibiotic out of the cell. Tet operon systems are widely available, such as the Tet operon used in the pcDNA™ 4/TO mammalian expression vector available from Invitrogen.

In one embodiment, a Tet responsive promoter is used to control expression of the nucleic acid sequences encoding the recombinase gene. In one embodiment, one or more of the helper virus genes (e.g. for adenovirus helper genes, any combination comprising one or more of E1A, E1B, E2A, E4 and VA), are under the control of an inducible expression system. As noted previously, E4 protein is reported to potentially be cytotoxic to cells (Ferrari et al., 1996, Journal of Virology 70: 3227-3234). As such, it may be desirable to be able to control the expression of E4 protein. Similarly it may be desirable to control the expression of E2A to alleviate any rep/cap gene amplification in the event of leaky Rep expression. In a further embodiment, a Tet responsive promoter is used to control expression of the nucleic acid sequences encoding one or more of the helper virus genes, for example the E2A, E4 and VA.

In one embodiment, the AAV vector producer cell line further comprises a nucleic acid sequence encoding a TetR gene. In a further embodiment, the TetR is TetR-KRAB. TetR-KRAB is a hybrid protein first described by Deuschle et al. (1995, Mol Cell Biol 15:1907-14) in which the Krüppel associated box (KRAB) domain from the human Kox1 zinc finger protein is fused to the C-terminus of the Tet repressor derived from Tn/10 of E. coli. This hybrid protein has the advantage of being able to silence the expression of a gene by binding tetO sites a long distance from the transcriptional start site. Promoter activity is restored upon administration of tetracycline or its derivative, which prevents binding of TetR-KRAB to the tetO sequences.

In one embodiment, exogenous control of the expression of the nucleic acid sequences encoding the recombinase gene and/or one or more the helper virus genes is provided by a “Tet-On” system. In this case, transcription of the nucleic acid sequences under transcriptional control is reversibly turned on in the presence of tetracycline or its derivative. Such inducible promoters contain arrays of Tet operon sequences upstream of a minimal promoter, mostly based on the CMV immediate early promoter. A Tet repressor protein, for example TetR-KRAB, is also required to be constitutively expressed in the AAV vector producer cell line.

Under normal cell culture conditions, the Tet-responsive promoter is bound by TetR repressor. Addition of doxycyclin to the cell growth medium, when the cells are at the correct density to initiate recombinant AAV vector production, destabilises the TetR and allows transcription of the recombinase gene and in further embodiments, also one or more of the helper virus genes under transcription control.

In one embodiment, the promoter is pCMV-TO2, a Pol II promoter. pCMV-TO2 contains a CMV enhancer and promoter upstream of 2× Tet operon sequences.

It will be understood by the person skilled in the art that the above embodiments relating to nucleic acid sequences introduced into the host cell genome are also applicable to the nucleic acid sequences comprised in the nucleic acid vector of the invention.

### Methods

According to one aspect of the invention, there is provided a method of producing a stable AAV vector producer cell line, comprising:

(a) introducing the nucleic acid vector described herein into a culture of mammalian host cells; and

(b) selecting within the culture for a mammalian host cell which has the nucleic acid sequences encoded on the vector integrated into an endogenous chromosome of the mammalian host cell.

The skilled person will be aware that introducing a nucleic acid vector into the host cell may be performed using suitable methods known in the art, for example, lipid-mediated transfection, microinjection, cell (such as microcell) fusion, electroporation or microprojectile bombardment. In one embodiment, the nucleic acid vector is introduced into the host cell by electroporation. It will be understood that the choice of method to use for introducing the nucleic acid vector can be chosen depending upon the type of mammalian host cell used.

The skilled person will be aware of methods in the art for integrating recombinant nucleic acid sequences encoding the proteins outlined previously into the host cell genome for generating an AAV vector producer cell line, for example, that disclosed in Yuan et al. (Yuan et al. (2011) Hum. Gene Ther. 22:613), which is incorporated herein by reference.

The nucleic acid sequences defined herein are introduced into the mammalian host cell using a single nucleic acid vector comprising a non-mammalian origin of replication and the ability to hold at least 25 kilobases (kb) of DNA.

According to one aspect of the invention, there is provided a nucleic acid vector comprising a non-mammalian origin of replication and the ability to hold at least 25 kilobases (kb) of DNA, characterized in that said nucleic acid vector comprises nucleic acid sequences encoding:

AAV rep and cap genes;

helper virus genes; and

a DNA genome of an AAV vector;

wherein the rep gene comprises an intron, said intron comprising a transcription termination sequence with a first recombination site located upstream and a second recombination site located downstream of the transcription termination sequence; and

wherein the nucleic acid sequences encoding the AAV rep and cap genes, each of the helper virus genes and the DNA genome of the AAV vector are arranged as individual expression cassettes within the nucleic acid vector.

Current methods for generating AAV vectors involve transient transfection of one or more of the viral genes (packaging genes or helper virus genes) and/or transgene into a host cell. However, many disadvantages have been associated with this method because it is costly and laborious such that it is not optimal for large-scale AAV vector production.

One solution would be to engineer a producer cell line that stably incorporates all of the genes required for production of a recombinant AAV vector and genetic elements required to control expression of said genes, particularly the rep gene (the genetic elements for controlling Rep and AAV helper gene expression outlined previously), to provide a simplified and scalable method for large-scale clinical grade manufacture of recombinant AAV for therapeutic use. However, such a producer cell is not available in the art.

By including all of the genes and regulatory elements in a nucleic acid vector, these can be inserted into the endogenous chromosomes of a mammalian host cell in one single step to produce an AAV vector producer cell. Therefore, the use of a nucleic acid vector, as proposed herein, would reduce selection pressure, reduce the silencing timeframe and allow for faster screening of potential producer cells. Furthermore, the genes required for AAV vector production included on the nucleic acid vector would all be integrated into the endogenous chromosomes of the mammalian host cell at a single locus. This would reduce the risk of individual viral genes becoming silenced and ensure that all the viral genes are evenly expressed.

Furthermore, by controlling expression of the Rep proteins known to be toxic to the cells, and in some embodiments, expression of one or more of the helper virus genes also, it is possible to establish AAV vector producer cell lines stably incorporating the packaging genes (rep and cap genes) and the helper virus genes, which has a normal growth rate and high stability so as to be able to reach the cell density required to produce AAV vectors in a large-scale bioreactor.

It will be understood that the nucleic acid vector construct may integrate more than once in the host cell genome at multiple different locations on different chromosomes (albeit with all of the encoded nucleic acid sequences present in a single locus). This may be beneficial for increasing expression levels of the transgenes and could potentially improve AAV titres.

The nucleic acid vector comprises nucleic acid sequences which encode the DNA genome of the recombinant AAV vector. When this nucleic acid sequence is replicated, it will become encapsidated within the AAV vector produced by the cell and therefore act as the AAV vector's “genome”. It will be understood that the DNA genome of the AAV vector is usually included on the “transfer plasmid” or “transfer vector” used in transient transfection methods. The transfer plasmid generally contains a promoter (such as CMV) operably linked to the transgene (and optionally a poly-adenylation [polyA] signal), between the two AAV ITRs. Therefore, reference to the “DNA genome of the AAV vector” as used herein refers to a nucleic acid sequence (usually encoding the transgene of interest) flanked by AAV ITRs. Thus, in one embodiment, the DNA genome of the AAV vector comprises one or more transgenes encoded between two AAV ITRs.

In one embodiment, multiple copies of the DNA genome of the AAV vector (i.e. the transfer vector) are included in the nucleic acid vector. Multiple copies of the transfer vector are expected to result in higher viral vector titre. For example, the nucleic acid vector may include two or more, such as three, four, five, six, seven, eight, nine or ten or more copies of the DNA genome of the AAV vector (i.e. the transfer vector).

The nucleic acid vector may contain one or a plurality of recombination site(s), in addition to the recombination sites in the termination intron. This would allow for target sequences to be integrated into the endogenous chromosomes of the mammalian host cell in a site-specific manner in the presence of a recombinase enzyme. The recombinase enzyme catalyses the recombination reaction between two recombination sites. In one embodiment, the recombination site is an att site (e.g. from lambda phage), wherein the att site permits site-directed integration in the presence of a lambda integrase. It will be understood that the reference to “lambda integrase” includes references to mutant integrases which are still compatible with the int/att system, for example the modified lambda integrases described in WO 2002/097059.

Each of the nucleic acid sequences are arranged as individual expression cassettes within the nucleic acid vector. The skilled person will understand that the cap gene is transcribed from the p40 promoter, which is located within the rep gene, such that the rep and cap genes (i.e. rep/cap gene) are not separated and present in a single expression cassette.

In one embodiment, the nucleic acid vector further comprises nucleic acid sequences encoding a recombinase gene, arranged as an individual expression cassette within the nucleic acid vector. In a further embodiment, the recombinase gene is a Cre recombinase gene. The Cre recombinase gene may be codon optimised (iCre).

In one embodiment, the nucleic acid vector further comprises a recombinase control system. In a further embodiment, the recombinase control system comprises a recombinase gene under the control of an inducible promoter and/or a steroid hormone receptor ligand-binding domain operably linked to the recombinase. In a further embodiment, the steroid hormone receptor ligand-binding domain is an estrogen receptor ligand binding-domain (ER). In one embodiment, the ER is operably linked upstream and downstream of the recombinase gene (i.e. the recombinase gene is flanked by ER). In one embodiment, the ER is ERT2.

In one embodiment, the nucleic acid vector additionally comprises an insulator, such as a chromatin insulator. The term “insulator” is well known in the art refers a class of DNA sequence elements that possess a common ability to protect genes from inappropriate signals emanating from their surrounding environment. (West et al., 2002, Genes Dev 16:271-288). In a further embodiment, the insulator (such as a chromatin insulator) is present between each of the nucleic acid sequences. The nucleic acid sequences in this context refers to the nucleic acid sequences in an expression cassette, such that the insulator sequences are present between expression cassettes. In one embodiment, an insulator is present between each of the expression cassettes.

When creating a large genetic construct that will be stably integrated into a host cell genome, it is often necessary to separate each element (i.e. expression cassette) of the construct with an insulator sequence. Transient transfection methods mask a major issue with multigene vectors, namely promoter interference from tandem promoters (Moriarity et al. (2013) Nucleic Acids Res. 41:e92). Moriarity et al. created a large stable construct that included two tandem copies of the cHS4 insulator (2×cHS4) between each expression element. The insulator sequence is a 1.2 kb cHS4 element from the chicken β-like globin gene cluster. These stretches of DNA act as potent enhancer blockers and was shown to overcome promoter interference. Without being bound by theory, two tandem copies of cHS4 is considered to alleviate promoter interference by providing binding sites for several proteins (CBP, CTCF and USF1) that maintain an open chromatin state by recruiting chromatin-modifying host factors (Yahata et al. (2007) J. Mol. Biol. 374:580).

Furthermore, the insulator elements can also be employed as a safety feature to alleviate issues arising from enhancer elements introduced into the host genome, which can sometimes elevate levels of host oncogenes. Rivella et al. (Rivella et al. (2000) J Virol. 74: 4679) also showed that transgene expression from integrated retroviruses was improved and methylation of the 5′ long terminal repeat, which is implicated in the silencing of integrated retroviruses, was reduced when cHS4 was incorporated upstream of the retroviral enhancer/promoter. For the reasons outlined above, it is, therefore, advantageous when creating the stable AAV vector producer BAC constructs to include an insulator between each expression element.

In one embodiment, the insulator has at least 90% sequence identity, for example at least 95% sequence identity, to the chicken (Gallus gallus) HS4 (cHS4) insulator sequence (for example see Genome Accession No. U78775.2, base pairs 1 to 1205). In a further embodiment, the insulator comprises two tandem cHS4 insulator sequences (approximately 2.4 kilobases), i.e. 2×cHS4.

In order to sequentially clone each expression cassette of the stable AAV vector producer construct (i.e. nucleic acid sequences encoding the genes required for generating an AAV vector producer cell line, respectively, as outlined previously) into a BAC along with 2×cHS4 elements, it is helpful to create a donor plasmid containing cHS4 downstream of rare cutting restriction sites, that would allow the cloning of each element next to the cHS4 before transferring both the element and the 2×cHS4 to the BAC. To facilitate this, the cloning site for the expression cassettes and the 2×cHS4 could be flanked by meganuclease sites, cut sites for restriction enzymes with long recognition sequences that rarely appear, even in lengthy constructs. The restriction sites of the meganuclease restriction enzymes I-SceI and PI-PspI could be present in the donor plasmid at the 5′ end of the expression cassette and 3′ of 2×cHS4 respectively. I-SceI and PI-PspI create compatible overhangs. This would allow each expression cassette cloned into the donor plasmid to be sequentially cloned, along with 2×cHS4, into the BAC downstream of the previous element plus cHS4 by so-called iBrick cloning as outlined in Liu et al. (Liu et al. (2014) PLoS One 9:e110852).

In a further embodiment, an insulator may be present between each of the helper virus genes (e.g. E1A, E1B, E2A, E4 and/or VA, or in HEK293 based producer cells, E2A, E4 and/or VA). This helps to prevent promoter interference (i.e. where the promoter from one transcription unit impairs expression of an adjacent transcription unit) between adjacent viral nucleic acid sequences. Without being bound by theory, this is also thought to help minimise the risk of recombination between viral sequences to generate replication-competent virus.

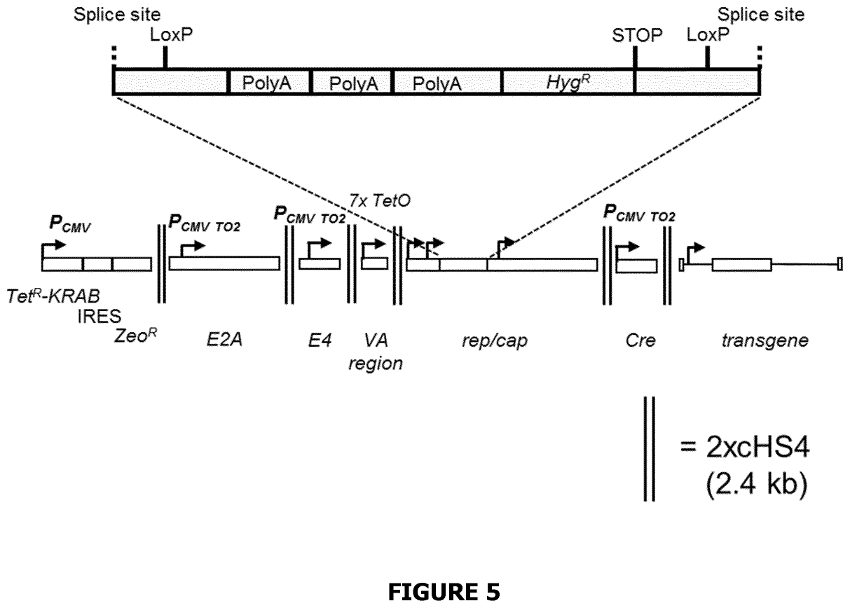

For example, in one exemplary embodiment, the nucleic acid vector comprises the following insert: a TetR-KRAB gene operably linked to a CMV promoter and a selection marker comprising IRES, an insulator (such as a chromatin insulator), an adenovirus helper gene E2A operably linked to a CMVTO2 promoter, an insulator (such as a chromatin insulator), an adenovirus helper gene E4 operably linked to a CMVTO2 promoter, an insulator (such as a chromatin insulator), 7 TetO sequences, an adenovirus helper gene VA operably linked to a promoter, an insulator (such as a chromatin insulator), a nucleic acid sequence encoding the AAV rep and cap genes with an intron between the P19 and P40 promoters containing a LoxP site followed by transcriptional terminator sequence followed by a hygromycine gene followed by a LoxP site, an insulator (such as a chromatin insulator), a nucleic acid sequence encoding Cre or ERT2-Cre-ERT2 operably linked to a CMVTO2 promoter, an insulator (such as a chromatin insulator), a nucleic acid sequence comprising a transgene operably linked to a promoter between two AAV ITRs and a multiple cloning site. An exemplary embodiment of the nucleic acid vector is provided schematically in FIG. 5.

In one embodiment, the nucleic acid vector is selected from: a bacterial artificial chromosome (BAC), a yeast artificial chromosome (YAC), a P1-derived artificial chromosome (PAC), fosmid or cosmid. In a further embodiment, the nucleic acid vector is a bacterial artificial chromosome (BAC). In one embodiment, the nucleic acid vector is a plasmid.

### Bacterial Artificial Chromosomes

The term “bacterial artificial chromosome” or “BAC” refers to a DNA construct derived from bacterial plasmids which is able to hold a large insert of exogenous DNA. They can usually hold a maximum DNA insert of approximately 350 kb. BACs were developed from the well characterised bacterial functional fertility plasmid (F-plasmid) which contains partition genes that promote the even distribution of plasmids after bacterial cell division. This allows the BACs to be stably replicated and segregated alongside endogenous bacterial genomes (such as E. coli). The BAC usually contains at least one copy of an origin of replication (such as the oriS or oriV gene), the repE gene (for plasmid replication and regulation of copy number) and partitioning genes (such as sopA, sopB, parA, parB and/or parC) which ensures stable maintenance of the BAC in bacterial cells. BACs are naturally circular and supercoiled which makes them easier to recover than linear artificial chromosomes, such as YACs. They can also be introduced into bacterial host cells relatively easily, using simple methods such as electroporation.

In one embodiment, the bacterial artificial chromosome comprises an oriS gene. In one embodiment, the bacterial artificial chromosome comprises a repE gene. In one embodiment, the bacterial artificial chromosome comprises partitioning genes. In a further embodiment, the partitioning genes are selected from sopA, sopB, parA, parB and/or parC. In a yet further embodiment, the bacterial artificial chromosome comprises a sopA and sopB gene.

BAC for use in the present invention may be obtained from commercial sources, for example the pSMART BAC from LUCIGEN™ (see Genome Accession No. EU101022.1 for the full back bone sequence). This BAC contains the L-arabinose “copy-up” system which also contains the oriV medium-copy origin of replication, which is active only in the presence of the TrfA replication protein. The gene for TrfA may be incorporated into the genome of bacterial host cells under control of the L-arabinose inducible promoter araC-P_{BAD} (see Wild et al. (2002) Genome Res. 12 (9): 1434-1444). Addition of L-arabinose induces expression of TrfA, which activates oriV, causing the plasmid to replicate to up to 50 copies per cell.

### Yeast Artificial Chromosomes

The term “yeast artificial chromosome” or “YAC” refers to chromosomes in which yeast DNA is incorporated into bacterial plasmids. They contain an autonomous replication sequence (ARS) (i.e. an origin of replication), a centromere and telomeres. Unlike BACs, the YAC is linear and therefore contains yeast telomeres at each end of the chromosome to protect the ends from degradation as it is passed onto host cell progeny. YACs can hold a range of DNA insert sizes; anything from 100-2000 kb.

### Pi-derived Artificial Chromosomes

The term “P1-derived artificial chromosome” or “PAC” refers to DNA constructs derived from the DNA of the P1-bacteriophage and bacterial F-plasmid. They can usually hold a maximum DNA insert of approximately 100-300 kb and are used as cloning vectors in E. coli. PACs have similar advantages as BACs, such as being easy to purify and introduce into bacterial host cells.

### Cosmids and Fosmids

The term “cosmid” refers to DNA constructs derived from bacterial plasmids which additionally contain cos sites derived from bacteriophage lambda. Cosmids generally contain a bacterial origin of replication (such as oriV), a selection marker, a cloning site and at least one cos site. Cosmids can usually accept a maximum DNA insert of 40-45 kb. Cosmids have been shown to be more efficient at infecting E. coli cells than standard bacterial plasmids. The term “fosmids” refers to non-mammalian nucleic acid vectors which are similar to cosmids, except that they are based on the bacterial F-plasmid. In particular, they use the F-plasmid origin of replication and partitioning mechanisms to allow cloning of large DNA fragments. Fosmids can usually accept a maximum DNA insert of 40 kb.

It will be understood that the nucleic acid sequences encoding the replication defective AAV vector may be the same as, or derived from, the wild-type genes, i.e. the sequences may be genetically or otherwise altered versions of sequences contained in the wild-type virus. Therefore, the viral genes incorporated into the nucleic acid vectors or host cell genomes, may also refer to codon-optimised versions of the wild-type genes.

It will be understood by the person in the art that embodiments relating to the nucleic acid vector are also applicable to the AAV vector producer cell. By way of example, and without limitation, the host cell genome may comprise insulators between nucleic acid sequences encoding the gene required for recombinant AAV vector production integrated therein.

Once transfected into the mammalian host cell, the nucleic acid vector will randomly integrate into the endogenous genome of the mammalian host cell. Therefore, the method additionally comprises selecting for the mammalian host cell in which the nucleic acids encoded on the nucleic acid vector have integrated (for example, using an antibiotic resistance selection marker, such as a zeocin resistance marker).

The skilled person will be aware of methods to encourage integration of the nucleic acid vector, for example, linearising the nucleic acid vector if it is naturally circular (for example, BACs, PACs, cosmids or fosmids). The nucleic acid vector may additionally comprise areas of shared homology with the endogenous chromosomes of the mammalian host cell to guide integration to a selected site within the endogenous genome. Furthermore, if recombination sites are present on the nucleic acid vector then these can be used for targeted recombination. For example, the nucleic acid vector may contain a LoxP site which allows for targeted integration when combined with Cre recombinase (i.e. using the Cre/lox system derived from P1 bacteriophage). Alternatively (or additionally), the recombination site is an att site (e.g. from lambda phage), wherein the att site permits site-directed integration in the presence of a lambda integrase. This would allow the viral genes to be targeted to a locus within the endogenous genome.

Other methods of targeted integration are well known in the art. For example, methods of inducing targeted cleavage of genomic DNA can be used to encourage targeted recombination at a selected chromosomal locus. These methods often involve the use of engineered cleavage systems to induce a double strand break (DSB) or a nick in the endogenous genome to induce repair of the break by natural processes such as non-homologous end joining (NHEJ) or repair using a repair template (i.e., homology directed repair or HDR).

Cleavage can occur through the use of specific nucleases such as engineered zinc finger nucleases (ZFN), transcription-activator like effector nucleases (TALENs), using the CRISPR/Cas9 system with an engineered crRNA/tracr RNA (‘single guide RNA’) to guide specific cleavage, and/or using nucleases based on the Argonaute system (e.g., from T. thermophilus, known as ‘TtAgo’, see Swarts et al. (2014) Nature 507 (7491): 258-261). Targeted cleavage using one of these nuclease systems can be exploited to insert a nucleic acid into a specific target location using either HDR or NHEJ-mediated processes. Therefore, in one embodiment, the method additionally comprises integrating the nucleic acid sequences encoded on the nucleic acid vector into the genome (i.e. an endogenous chromosome) of the mammalian host cell using at least one nuclease, wherein at least one nuclease cleaves the genome of the mammalian host cell such that the nucleic acid sequences are integrated into the genome of the cell. In a further embodiment, the nuclease is selected from the group consisting of a zinc finger nuclease (ZFN), a TALE nuclease (TALEN), a CRISPR/Cas nuclease system and combinations thereof.

An embodiment of a nucleic acid vector of the present invention is represented schematically in FIG. 5.

### Additional Components

In one embodiment, the nucleic acid vector comprises a selectable marker. In embodiments wherein the termination intron comprises a selectable marker, the selectable marker may be a different selectable marker in addition to the selectable marker in the termination intron. The selectable marker allows the cells which have incorporated the nucleic acid vector sequences to be selected. In a further embodiment, the selectable marker is a drug resistance gene, such as an antibiotic resistance gene, e.g. a zeocin, kanamycin or puromycin resistance gene, in particular a zeocin (ZeoR) resistance gene. In a yet further embodiment, the zeocin resistance gene is derived from the Streptoalloteichus hindustans ble gene, for example see Genome Accession No. X52869.1 from base pairs 3 to 377.

The natural phenomenon of gene amplification has been exploited in the biopharmaceutical industry as a way of increasing the titre of a recombinant product produced by a cell line. Where a recombinant gene has been integrated into the host cell's genome, the copy number of the recombinant gene and concomitantly the amount of recombinant protein expressed can be increased by selecting for cell lines in which the recombinant gene has been amplified after integration into the host cell genome. Therefore, in one embodiment, the selectable marker is an amplifiable selection marker.

Gene amplification may be induced by stably transfecting a host cell with an amplifiable selection marker gene. The stably transfected host cells are subjected to increasing concentrations of a toxic drug, which is known to inhibit the amplifiable selection marker. For example, the transfected cells may be cultured in a medium which contains the toxic drug at a concentration to achieve killing of greater than 98% of the cells within 3 to 5 days after plating the parent cells (i.e. non-transfected cells) in medium containing the toxic drug. Through such inhibition, populations of cells can be selected that have increased expression levels of the amplifiable selection marker and, consequently, resistance to the drug at the concentration employed.

As noted above, the nucleic acid vector disclosed herein allows all of the expression cassettes contained therein (i.e. nucleic acid vector DNA) to be integrated together at a single locus within the host cell genome. As the process of gene amplification causes amplification of the amplifiable selection marker gene and surrounding DNA sequences, the remaining DNA sequences in the integrated nucleic acid vector DNA will also be amplified. In this way, it is possible to provide a process for gene amplification of viral vector genes stably integrated into a host cell genome.

Each amplifiable selection marker has an associated selection agent (i.e. a toxic drug), which is added to the cell culture media during amplification and selection regimes. Suitable amplifiable selection marker/selection agent combinations include adenosine deaminase/deoxycoformycin, aspartate transcarbamylase/N (phosphoacetyl)-L-aspartate, dihydrofolate reductase/methotrexate, glutamine synthetase/methionine sulphoximine, metallthionein-I/heavy metal.

In one embodiment, the amplifiable selection marker gene and/or the selectable marker is provided in an expression cassette.

In one embodiment, the amplifiable selection marker is dihydrofolate reductase (DHFR). The DHFR selection method involves incorporating the dhfr gene (amplifiable selection marker gene) to the nucleic acid vector thereby inducing a DHFR selection pressure to the other expression cassettes within the nucleic acid vector. The host cell is transfected with the nucleic acid vector and grown in the presence of increasing concentrations of DHFR inhibitor, methotrexate (MTX), to select for cells which have amplified the dhfr gene integrated into the host genome and concomitantly, the remaining integrated nucleic acid vector DNA.

In one embodiment, the dhfr gene comprises at least 60% sequence identity, such as at least 70%, 80%, 90% or 100% sequence identity to Genome Accession No. NM_010049.3

In another embodiment, the amplifiable selection marker is glutamine synthetase (GS). The GS selection method involves incorporating the gs gene to the nucleic acid vector, thereby inducing a GS selection pressure to the other expression cassettes within the nucleic acid vector. The host cell is transfected with the nucleic acid vector and grown in the presence of increasing concentrations of GS inhibitor methionine sulfoximine (MSX) to select for cells which have amplified the gs gene integrated into the host genome and concomitantly, the remaining integrated nucleic acid vector DNA.

The expression construct comprising nucleic acid sequences of the gs gene may contain nucleic acid sequences of expression constructs encoding gs gene known in the art (e.g. WO874462, which the sequences contained therein are incorporated herein by reference).

By using the amplifiable selection marker and associated selection agent in this way, followed by a culture period to allow the selection of cells that grow in the new (increased) concentration of the associated agent, the area of the genome harbouring the selection pressure can amplify, thereby increasing the copy number of the amplifiable selection marker. Consequently, when the expression cassettes of the nucleic acid vector comprising the viral genes are integrated into the host genome at a single locus together with an expression cassette comprising the amplifiable selection marker gene, these expression cassettes are also amplified. Therefore, the cell lines that grow through such rounds of amplification and selection are then screened on titre/yield and the best clone is selected for subsequent production of the AAV vector.

In a preferred embodiment, the host cell is negative for the amplifiable selection marker. That is to say, that the endogenous chromosome of the host cell does not comprise an endogenous amplifiable selection marker gene. For example, when using DHFR as the amplifiable selection marker, it is preferable to employ DHFR-negative host strains, such as CHO DG44 or CHO DUX-B11.

However, the invention is not limited by the choice of a particular host cell line. Any cell line which has a rapid rate of growth (i.e., a doubling time of 12 hours or less) and which is capable of amplifying the amplifiable selection marker gene at a reasonable rate without amplification of the endogenous amplifiable selection marker gene at a similar or higher rate may be used in the methods of the present invention.

Cell lines transduced with the dominant marker (i.e. exogenous amplifiable selection marker) are identified by determining that the ability of the cell to grow in increasing concentrations of the selection agent correlates with an increase in the copy number of the amplifiable selection marker (this may be measured directly by demonstrating an increase in the copy number of the amplifiable marker by Southern blotting or indirectly by demonstrating an increase in the amount of mRNA produced from the amplifiable marker by Northern blotting, or qPCR).

Where a host cell comprises an endogenous amplifiable selection marker gene, the nucleic acid vector may further comprise a nucleic acid sequence encoding a selectable marker in addition to the amplifiable selection marker. This circumvents the problem of amplification of the endogenous amplifiable selection marker gene during selection with the associated selection agent. The host cells are transfected with a nucleic acid vector comprising an amplifiable selection marker as well as a selectable marker. The transfected host cells are first selected for the ability to grow in the antibiotic of the selectable marker, such as zeocin or hygromycin p. The cells are then selected for the ability to grown in increasing concentrations of the selection agent, such as MTX.

In one embodiment, the nucleic acid vector comprises a polyA signal additionally to the polyA signal present in the termination intron. The use of a polyA signal has the advantage of protecting mRNA from enzymatic degradation and aiding in translation. In a further embodiment, the polyA signal is obtained from or derived from SV40, Bovine Growth Hormone and/or Human Beta Globin. In one embodiment, the polyA signal is derived from the SV40 early polyA signal (for example, see Genome Accession No. EF579804.1, base pairs 2668 to 2538 from the minus strand). In one embodiment, the polyA signal is derived from the Human Beta Globin polyA signal (for example, see Genome Accession No. GU324922.1, base pairs 3394 to 4162).

In one embodiment, the nucleic acid vector additionally comprises an intron sequence additionally to termination intron. The use of an intron downstream of the enhancer/promoter region and upstream of the cDNA insert (i.e. the transgene) is known to increase the level of expression of the insert. In a further embodiment, the intron sequence is a Human Beta Globin Intron or the Rabbit Beta Globin Intron II sequence. In one embodiment, the Human Beta Globin Intron is derived from the sequence available at Genome Accession No. KM504957.1 (for example from base pairs 476 to 1393). In one embodiment, the Rabbit Beta Globin Intron II is derived from the sequence available at Genome Accession No. V00882.1 (for example, from base pairs 718 to 1290).

In one embodiment, the nucleic acid vector additionally comprises a woodchuck hepatitis virus post-transcriptional regulatory element (WPRE). The presence of WPRE has been shown to enhance expression and as such is likely to be beneficial in attaining high levels of expression. In a further embodiment, the WPRE is derived from the sequence available at Genome Accession No. J04514.1 (for example, from base pairs 1093 to 1684).

In one embodiment, the nucleic acid vector additionally comprises an internal ribosome entry site (IRES). An IRES allows for translation initiation in an end-independent manner. An IRES is a structured RNA element that is usually found in the 5′-untranslated region (UTR) of viruses downstream of the 5′-cap (which is required for the assembly of the initiation complex). The IRES is recognized by translation initiation factors and allows for cap-independent translation. In a further embodiment, the IRES is derived from the Encephalomyocarditis virus (EMCV) genome (for example, see Genome Accession No. KF836387.1, base pairs 151 to 724).

In one embodiment, the nucleic acid vector additionally comprises a Multiple Cloning Site (MCS). An MCS is a short segment of DNA within the nucleic acid vector which contains multiple restriction sites (for example, 10, 15 or 20 sites). These sites usually occur only once within the nucleic acid vector to ensure that the endonuclease only cuts at one site. This allows for the viral genes to be easily inserted using the appropriate endonucleases (i.e. restriction enzymes).

It will be understood by a person skilled in the art that the expression cassettes may be arranged in any order within the nucleic acid vector.

The nucleic acid sequences may be introduced into the nucleic acid vector sequentially. This allows for selection after each integration to ensure that all of the required nucleic acid sequences are successfully integrated into the nucleic acid vector. Alternatively, at least two or more of the nucleic acid sequences are introduced into the nucleic acid vector simultaneously.

It will be understood that the additional genes described herein may be introduced into the nucleic acid vector by standard molecular cloning techniques known in the art, for example using restriction endonucleases and ligation techniques. Furthermore, the nucleic acid vector, in particular BACs, PACs, fosmids and/or cosmids, may be introduced into bacterial host cells (such as E. coli cells, in particular the E. coli strain DH10B) by standard techniques, such as electroporation.

According to a further aspect of the invention, there is provided an AAV vector producer cell obtained by the methods defined herein.

The cell line obtained using the methods defined herein may be used to produce a high titre of AAV vector. Viral titre may be measured by quantitative PCR (qPCR), which provides the genome copy number of AAV particles, and by ELISA which provides the TCID50 measure of infectious virus titre. By comparing the two measurements, the efficiency of transduction with the AAV batch can be determined.

References herein to the term “high titre” refer to an effective amount of AAV vectors which is capable of transducing a target cell, such as a patient cell. In one embodiment, a high titre is in excess of 106 TU/ml without concentration (TU=transducing units).

In one embodiment, the methods defined herein are scalable, so they can be carried out in any desired volume of culture medium, e.g., from 10 ml (e.g., in shaker flasks) to 10 L, 50 L, 100 L, or more (e.g. in bioreactors such as wave bioreactor systems and stirred tanks).

According to a further aspect of the invention, there is provided a method of producing a replication defective AAV vector, comprising:

(a) introducing the nucleic acid vector described herein into a culture of mammalian host cells; and

(b) selecting within the culture for a mammalian host cell which has the nucleic acid sequences encoded on the vector integrated into an endogenous chromosome of the mammalian host cell; and

(c) further culturing the selected mammalian host cell under conditions in which the replication defective AAV vector is produced.

It will be understood by the skilled person that the conditions used in the method described herein will be dependent upon the host cell used. Typical conditions, for example the culture medium or temperature to be used, are well known in the art. In one embodiment, culturing is performed by incubating the mammalian host cell under humidified conditions. In a further embodiment, the humidified conditions comprise incubating the transfected cells at 37° C. at 5% CO2. In one embodiment, culturing is performed using a culture medium selected from: Dulbecco's modified Eagle's medium (DMEM) containing 10% (vol/vol) foetal bovine serum (FBS), serum-free UltraCULTURE™ medium (Lonza, Cat. No. 12-725F), or FreeStyle™ Expression medium (Thermo Fisher, Cat. No. 12338 018).

Appropriate culturing methods are well known to a person skilled in the art. For example, the cell may be cultured in suspension and/or in animal component-free conditions. In one embodiment, the cell is suitable for culturing in any volume of culture medium, from 10 ml (e.g. in shaker flasks) to 10 L, 50 L, 100 L, or more (e.g. in bioreactors).

As described herein, use of the claimed invention reduces the cost of plasmid manufacture, reduces requirement for transfection reagents (e.g. Polyethylenimine [PEI]), reduces the amount of Benzonase treatment required (there is a reduced amount of DNA in the viral harvest, therefore less Benzonase is needed to remove the excess in downstream processing) and reduces costs of testing (there is no need to test for residual plasmid in the viral product). All of these advantages may be considered as aspects of the invention.

In one embodiment, the method additionally comprises isolating the replication defective AAV vector. For example, in one embodiment the isolating is performed by using a filter. In a further embodiment, the filter is a low-protein binding membrane (e.g. a 0.22 μm low-protein binding membrane or a 0.45 μm low-protein binding membrane), such as polyvinylidene fluoride (PVDF) or polyethersulfone (PES) artificial membranes.

Once inside the mammalian host cell, the nucleic acid sequences present on the nucleic acid vector may integrate into a random location within the endogenous genome. The integration step may be encouraged as described herein before, for example using linearisation and/or areas of shared homology. Recombination sites may also be used for targeted recombination.

If the target genes are integrated into the endogenous chromosomes with a selective marker, such as an antibiotic resistance gene, then the method may additionally comprise selecting for the mammalian host cells in which the viral nucleic acids have successfully integrated.

Once isolated, the AAV vectors may be concentrated for in vivo applications. Concentration methods include, for example, ultracentrifugation, precipitation or anion exchange chromatography. Ultracentrifugation is useful as a rapid method for AAV vector concentration at a small scale. Alternatively, anion exchange chromatography (for example using Mustang Q anion exchange membrane cartridges) or precipitation (for example using PEG 6000) are particularly useful for processing large volumes of AAV vector supernatants.

According to a further aspect of the invention, there is provided a replication defective AAV vector obtained by the method defined herein.

It will be noted that embodiments of the nucleic acid vectors used in the methods described herein is also taken as embodiments of the AAV vector producer cells and vice versa in so far as the embodiments relate to features of the nucleic acid vector which are integrated into the host genome of the AAV vector producer cell via the respective method.

The invention will now be described in further detail with reference to the following, non-limiting Examples.

### EXAMPLES

### Example 1

The following modifications were made to the standard packaging plasmid (rep and cap genes), helper virus plasmid (E2A, E4 and VA genes) and transfer vector plasmid (transgene flanked by ITR sequences, encoding the DNA genome of the recombinant AAV vector) used for AAV vector production.

### Design of Termination Intron Containing Transcriptional Termination Sequences Flanked by LoxP Sites

To recreate the 3 SV40 polyA sequences in silico, the early polyA sequence was copied from complete SV40 genome (Accession number J02400.1) and 3 copies were ligated in tandem.

A HSV TK promoter sequence was placed upstream of a hygromycin resistance gene sequence (GenBank accession number U40398.1). The HSV TK promoter and hygromycin resistance gene was placed downstream of the 3 SV40 poly A sequences.

The sequence of the LoxP sites was taken directly from Qiao et al. (2002, Journal of Virology 76: 13015-13027). This sequence was pasted at the 5′ and 3′ ends of the 3× polyA-HygR fragment resulting in LoxP sites flanking the transcriptional terminators in the same orientation.

The human chorionic gonadotrophin (hCG) intron 1 sequence was obtained from the hCG gene 5 beta subunit (accession number X00265.1). LoxP flanked 3× SV40 polyA-HygR sequence was inserted in the hCG intron at base 196. The length of the complete termination intron sequence was 3120 bp.

### Cloning of Termination Intron into AAV Rep Gene

The termination intron was inserted into rep2cap2 and rep2cap5 expression plasmids at position 1022 relative to the wild-type AAV2 genome sequence (GenBank accession number: J01901). This is within the rep gene downstream of promoter P19. This position contains a consensus splice donor/acceptor sequence for an intron to be inserted into (AAG/G).

### Design of Codon Optimised Cre (iCre) Sequence

A codon improved variant of Cre, named iCre, was designed by Shimshek et al. (2002 Genesis 32: 19-26) and was shown to restore nearly 2× the β-galactosidase activity of LacZ containing a “floxed” stop codon compared to wild type Cre when transiently transfected into CV1-5B cells (Casanova et al., 2002 Genesis 34: 208-214).

Cre sequence was obtained from GenBank Accession number DQ023272. iCre sequence was obtained from GenBank accession no. AY056050.

### Cloning of CMV-TO2 Promoter of Genes for Tet Induced Expression

In addition to regulating Rep expression, it may be necessary to regulate the expression of the Adenovirus 2 helper genes in the BAC (E2A, E4 and VA with HEK293 cells) as these are potentially toxic to the cells as well (Ferrari et al., 1996, Journal Of Virology 70: 3227-3234). Therefore, the E2A DNA binding protein coding region and the E4 region containing all 6 E4 ORFs were cloned, respectively, downstream of PCMV-TO2. However, VA, being transcribed from a Pol III promoter, would not transcribe correctly if put downstream of PCMV-TO2. Therefore, several TetO sequences were placed upstream of the native VA promoter and the binding of TetR-KRAB to these sites should inhibit transcription in the absence of doxycycline.

Accordingly, the CMV-TO2 promoter was cloned upstream of Cre, iCre, ERT2-Cre-ERT2, ERT2-iCre-ERT2, Adenovirus 2 E2A, and E4 individually using Gibson assembly. Furthermore, 7× TetO sequences were cloned upstream of Adenovirus 2 VA using Gibson assembly.

### Design of TetR-KRAB

The expression of Cre is under the control of a conditional promoter (PCMV-TO2) that, under normal cellular conditions, is bound by the transcriptional repressor TetR-KRAB. Addition of doxycycline to the cell growth medium, when the cells are at the correct density to initiate rAAV production, destabilises the TetR-KRAB repressor and allows transcription of the Cre gene. Subsequently, the Cre recombinase will splice out the LoxP flanked transcriptional terminators in a recombinant intron in the AAV rep gene, allowing transcription of the rep gene. This system means that Rep is only expressed in cells upon addition of doxycycline to the medium, alleviating the toxicity to the cells until they reach the required density suitable for AAV vector production.

A sequence encoding the KRAB domain from the human Kox1 gene cloned at the 3′ end of codon optimised TetR gene by Gibson assembly to convert the TetR gene under the control of a PCMV promoter into a TetR-KRAB gene.

### Design of Tamoxifen Regulated ERT2Cre/iCreERT2

Any leaky Cre expression under the control of Tet conditional expression system could still effectively remove the transcriptional terminators from the recombinant AAV rep gene, and reduce the viability of the stable cells in culture. Therefore, as a safeguard against this, an additional layer of Cre recombinase control was added by modifying the Cre gene by flanking it with ERT2 domains as described in Casanova et al. (2002, Genesis 34: 208-214). These ERT2 domains at the N and C terminus of the protein inhibit Cre from entering the nucleus until 4-hydroxy-tamoxifen is added to the cell culture medium, giving a level of control over Cre activity at the protein level that can relieve the effects of any leaky expression at the transcriptional level.

ERT2 domain sequences (Wagner J, Metzger D, Chambon P (1997) Biochem Biophys Res Commun 237:752-757) were cloned at the 5′ and 3′ ends of Cre and iCre genes, downstream of the CMVTO2 conditional promoter using Gibson assembly.

### Example 2

### Transient Transfection Method to Test Ability of Cre to Remove the Termination Intron from Rep Gene

A termination intron containing 3 SV40 poly A transcriptional termination sites and a hygromycin resistance gene, all flanked by LoxP sites, was inserted into the AAV2 rep sequence downstream of the P19 promoter in our rep2/cap2 and rep2/cap5 expression plasmids as noted above. The termination intron should inhibit the expression of Rep proteins in cells transfected with these plasmids unless Cre is also expressed in the cells. Co-transfection of a Cre expression plasmid should recombine the 2 LoxP sites, splicing out the transcriptional terminators and allow the RNA polymerase to read through the remaining intron. Several plasmids had been previously constructed in which various Cre variants (wild type Cre, iCre, ERT2-Cre-ERT2 and ERT2-iCre-ERT2) were cloned downstream of the CMVTO2 promoter, which is constitutively active in cells in the absence of a Tet repressor protein. The ERT2 flanked Cre proteins also require 4-hydroxy-tamoxifen to to act as a ligand that allows them to enter the nucleus.

The ability of the termination intron present in the recombinant rep intron was tested for their ability to stop Rep protein from being produced. Additionally, the various Cre expression plasmids were tested for their ability to restore Rep expression in cells that they are co-transfected into.

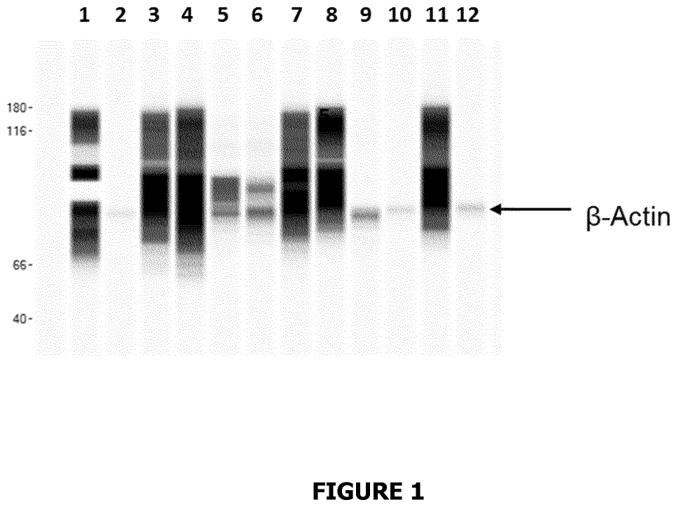

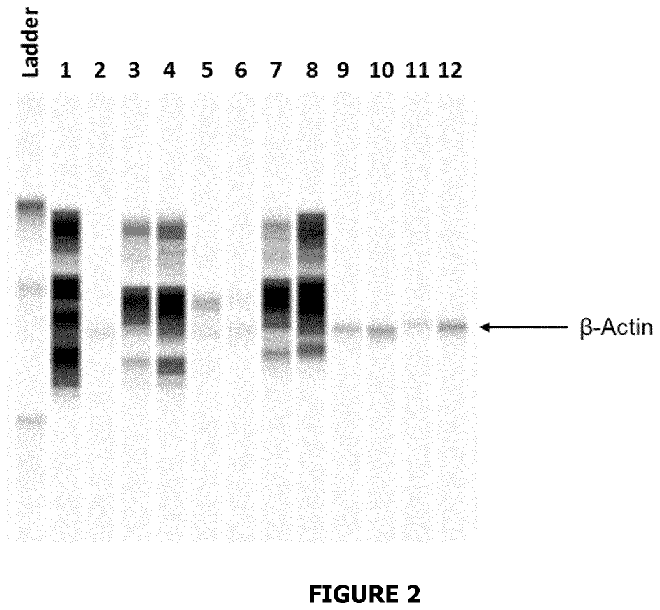

Adherent HEK 293T cells were disaggregated with TrypLE Express, re-suspended in DMEM+10% FCS, counted using a NucleoCounter NC-250 and diluted to 2×10⁵ cells/ml. The cells were plated in a 24-well plate, 1 ml per well, and incubated overnight at 37° C. The following day, the cells in the 24-well plate were sub-confluent. Wells of cells were co-transfected with the combinations of plasmids listed below (with reference to FIGS. 1 and 2), each plasmid was used at 0.5 μg/well. Where used, 4-hydroxytamoxifen was added to the cell growth medium at 1 μM (1:1000 dilution of 1 mM stock dissolved in ethanol).

### FIG. 1

### 1. pG.AAV2.R2C2+pG3.Ad2 Helper

2. pG.AAV2.R2C2-hCG intron 3× pA Hyg+pG3.Ad2 Helper
3. pG.AAV2.R2C2-hCG intron 3× pA Hyg+pG3.Ad2 Helper+pG3.CMVTO2-Cre
4. pG.AAV2.R2C2-hCG intron 3× pA Hyg+pG3.Ad2 Helper+pG3.CMVTO2-iCre
5. pG.AAV2.R2C2-hCG intron 3× pA Hyg+pG3.Ad2 Helper+pG3.CMVTO2-ERT2-Cre-ERT2
6. pG.AAV2.R2C2-hCG intron 3× pA Hyg+pG3.Ad2 Helper+pG3.CMVTO2-ERT2-iCre-ERT2
7. pG.AAV2.R2C2-hCG intron 3× pA Hyg+pG3.Ad2 Helper+pG3.CMVTO2-ERT2-Cre-ERT2+4-hydroxytamoxifen
8. pG.AAV2.R2C2-hCG intron 3× pA Hyg+pG3.Ad2 Helper+pG3.CMVTO2-ERT2-iCre-ERT2+4-hydroxytamoxifen
9. pG.AAV2.R2C2-hCG intron 3× pA Hyg (negative control)
10. pG3.Ad2 Helper GSK (negative control)
11. pG.AAV2.R2C2-hCG intron 3× pA Hyg+pG3.Ad2 Helper GSK+pG3.CMVTO2-Cre+1 μM 4-hydroxytamoxifen (in case 4-hydroxytamoxifen has any effect in the absence of ERT2)
12. Untransfected cells.

### FIG. 2

### 1. pG2.AAV5.R2C5+pG3.Ad2 Helper

2. pG2.AAV5.R2C5-hCG intron 3× pA Hyg+pG3.Ad2 Helper
3. pG2.AAV5.R2C5-hCG intron 3× pA Hyg+pG3.Ad2 Helper+pG3.CMVTO2-Cre
4. pG2.AAV5.R2C5-hCG intron 3× pA Hyg+pG3.Ad2 Helper+pG3.CMVTO2-iCre
5. pG2.AAV5.R2C5-hCG intron 3× pA Hyg+pG3.Ad2 Helper+pG3.CMVTO2-ERT2-Cre-ERT2
6. pG2.AAV5.R2C5-hCG intron 3× pA Hyg+pG3.Ad2 Helper+pG3.CMVTO2-ERT2-iCre-ERT2
7. pG2.AAV5.R2C5-hCG intron 3× pA Hyg+pG3.Ad2 Helper+pG3.CMVTO2-ERT2-Cre-ERT2+4-hydroxytamoxifen
8. pG2.AAV5.R2C5-hCG intron 3× pA Hyg+pG3.Ad2 Helper+pG3.CMVTO2-ERT2-iCre-ERT2+4-hydroxytamoxifen

### 9. pG3.Ad2 Helper

10. pG2.AAV5.R2C5-hCG intron 3× pA Hyg (negative control)
11. pG2.AAV5.R2C5-hCG intron 3× pA Hyg+1 μM 4-hydroxytamoxifen (negative control)
12. Untransfected cells

The plasmids were all added to tubes containing 75 μl OPTI MEM. To each tube, a further 75 μl OPTI MEM containing 2 μl of PEI Pro was added. The tubes containing the transfection mixes were mixed by vortexing briefly and incubated at room temperature for 10 minutes. Following this, the growth media was removed from the cells in the 24-well plate and replaced with 150 μl of each of the transfection mixes. Following a 10 minute incubation, the wells were topped up with 1 ml of DMEM+10% FCS and the plate incubated at 37° C. for 24 hours. The following day, the media was removed from the wells containing the transfected cells and the cells were lysed by addition of 300 μl per well of M-PER mammalian protein extraction reagent containing 1×Halt™ protease inhibitor and pipetted up and down. The tubes were then spun at 14,000×g for 5 minutes to remove the cell debris and the lysates transferred to new tubes. The total protein concentration of each sample was determined using a Pierce BCA protein assay kit. A BSA standard curve was set up as described in the kit manual. A volume of 25 μl of each standard and sample was added to a flat-bottomed 96-well plate in triplicate and 200 μl of the BCA working reagent was added to each well. The plate was incubated at 37° C. for 30 minutes and the absorbance was then measured at a wavelength of 562 nm using a FlexStation 3. The samples were all normalised to a concentration of 0.25 mg/ml using M-PER lysis buffer.

The samples were prepared for running using the Peggy Sue size protocol. A volume of 1 μl of reconstituted Fluorescent master mix from the Standard Pack 1 was added to 4 μl of each of the samples in eppendorf tubes. The samples and the biotinylated ladder from the Standard Pack 1 were then vortexed and denatured by heating at 95° C. for 5 minutes. The samples were then spun down and placed on ice. The primary αAAV Rep antibody (diluted 1:100) and the primary αβ-Actin antibody (diluted 1:500) were combined in Antibody Diluent 2. The samples, antibody, luminol/peroxide mix, primary and secondary antibody (αMouse, used neat), streptavidin-HRP, and the separation and stacking matrixes were loaded on a plate from the Peggy and Sally Sue 12-230 kDa separation kit.

Owing to technical difficulties, the plate needed to be stored at 4° C. over the weekend and it was run on the following Monday on the Peggy Sue. The plate was mounted on the Peggy Sue with the lid on and the positions of each sample and reagent entered into the Compass for SW software. A new set of capillaries from the Peggy and Sally Sue 12-230 kDa separation kit was installed and the Peggy Sue was then run over night.

As expected, all 4 splice variants of Rep2 were detected in the lysate of cells co-transfected with the Ad2 helper plasmid and either rep2/cap2 or rep2/cap5 expression plasmids. Lysate of cells co-transfected with the Ad2 helper plasmid and either pG.AAV2.R2C2-hCG intron 3× pA Hyg or pG2.AAV5.R2C5-hCG intron 3× pA Hyg did not contain any detectable Rep2 protein. They did contain the β-Actin band, proving that this was not due to a capillary failure on the Peggy Sue. This shows that the intron containing the LoxP-flanked 3× SV40 polyA transcriptional terminators and Hygromycin resistance gene downstream of the P19 promoter in rep is effective at inhibiting expression of Rep.

Lysate of cells co-transfected with the Ad2 helper plasmid and either pG.AAV2.R2C2-hCG intron 3× pA Hyg or pG2.AAV5.R2C5-hCG intron 3× pA Hyg along with either the Cre or iCre expression plasmids contained high levels of Rep2 protein. This shows that the removal of the 3× SV40 polyA transcriptional terminators and Hygromycin resistance gene from the hCG intron downstream of P19 in rep by Cre recombination of the LoxP sites restored expression. In each case, the iCre expression plasmid seemed to be slightly more effective at restoring Rep2 expression than the wild type Cre expression plasmid.

Lysate of cells co-transfected with the Ad2 helper plasmid and either pG.AAV2.R2C2-hCG intron 3× pA Hyg or pG2.AAV5.R2C5-hCG intron 3× pA Hyg along with either the ERT2-Cre-ERT2 or ERT2-iCre-ERT2 expression plasmids, in the absence of 4-hydroxytamoxifen, only expressed trace amounts of Rep2 protein. This was likely due to residual activity of the ERT2-flanked Cre proteins that were possibly at such high levels in the cell and, therefore, small amounts were able to enter the cell nuclei in the absence of ligand and cleave the transcriptional terminators from the intron in rep2. Alternatively, phenol red in the DMEM cell culture medium can act as a weak estrogen receptor ligand. Cells transfected with these plasmids in the presence of 1 μM 4-hydroxytamoxifen in the growth medium had Rep2 expression levels similar to those cells transfected with the Cre and iCre expression plasmids. The addition of 4-hydroxytamoxifen therefore restored full Cre activity to the ERT2-flanked Cre proteins. In each case, cells transfected with the plasmids expressing ERT2-iCre-ERT2 had lower residual Rep2 expression in the absence of 4-hydroxytamoxifen, and higher levels of Rep2 expression in the presence of 4-hydroxytamoxifen than cells transfected with EET2-Cre-ERT2.

No Rep2 was detected in the negative untransfected cell lysate. The β-Actin antibody showed that the loading was not completely even but good enough to show that any absence of Rep2 expression was not due to the failure of the capillary. In capillaries where the lysate contained Rep2, the lower Rep2 splice variants obscured the β-Actin signal.

This experiment shows that the LoxP-flanked transcriptional terminators in the termination intron cloned into the AAV2 rep gene were effective at inhibiting Rep2 expression, and that Cre activity in the cells could restore this Rep2 expression. This experiment also showed that the activity of the ERT2-flanked Cre proteins was conditional on 4-hydroxytamoxifen though they did have low levels of residual activity in the absence of this ligand. Also, the codon improved iCre restored slightly higher Rep2 expression than wild type Cre.

A stably transfected cell line containing this recombinant rep gene would not express Rep until Cre expression was activated. The Cre gene, under the control of a conditional promoter, could therefore be used to switch on Rep expression, and initiate AAV vector production, once the cells have reached optimal density in a bioreactor, alleviating the toxicity associated with Rep expression until they reach that point.

### Example 3

### Test to Determine Ability of Adenovirus 2 Helper Genes with Conditional Promoters to Sustain AAV Vector Production and to Determine if Cre-dependent Rep/Cap is Capable of Producing Functional AAV Vector

A stable BAC construct was designed and built for recombinant AAV vector production in which the native promoters of the Adenovirus 2 E2A and E4 genes were replaced with the conditional promoter CMV-TO2. The BAC also carries the DOX-sensitive “tet-on” transcriptional repressor gene TetR-KRAB downstream of the constitutive promoter P_{CMV}. Cells transfected with this construct would constitutively express TetR-KRAB which binds the CMVTO2 promoters upstream of the E2A and E4 ORFs, blocking their transcription until doxycycline is added to the cell growth medium. As TetR-KRAB is also capable of blocking transcription from Pol. III promoters by binding to nearby Tet operon sequences, 7 Tet operons were placed upstream of the VA native Pol. III promoter so expression of this functional RNA would also be blocked under normal conditions.

It was unknown if replacing the endogenous promoters of E2A and E4 would disrupt their expression and therefore inhibit their ability to provide helper functions to the AAV Rep and Cap proteins in cells during vector production.

It was important to test the ability of the BAC construct comprising all the Ad2 helper genes with conditional promoters and the TetR-KRAB gene (CreBAC6) to provide helper function during transient AAV vector production in HEK293 T cells compared to a similar construct in which all the Ad2 helper genes retained their endogenous promoters (BAC6). For CreBAC6 to be active as a helper, it would require addition of DOX to the cell growth medium. Helper function was tested by transfecting flasks of suspension adapted HEK293 cells with these BACs along with plasmids carrying AAV rep/cap (pG2.AAV2.R2C5-intron) and the EGFP expression transfer vector (pG.AAV2.C.GFP.P2a.fLuc.W6.ssb) used in the transient 3-plasmid system of vector production and incubating them. Any vector produced could be harvested by lysing the cells and used to transduce recipient CHO cells. Level of transduction, and therefore level of functional AAV vector production was assessed by measuring the percentage of GFP positive cells by flow cytometry.

### AAV Production

A flask of HEK293Tsa cells (HEK293Tsa cells as used in this application refers to suspension adapted HEK293T cells) were transfected with the standard plasmids of the 3-plasmid system for transient rAAV5 production (pG3.Ad2 Helper GSK, pG2.AAV5.R2C5-intron and pG.AAV2.C.GFP.P2a.fLuc.W6.ssb) as a known positive control.

HEK293Tsa cells were seeded in 250 ml shaker culture flasks at 2×10⁶ cells per ml, 60 ml per flask in BalanCD HEK293 media, 2% Glutamax, 0.1% Pluronic F-68. Due to the large size and limited yield of the BACs to be tested for helper function, it was not possible to transfect cells at the usual molar ratio for a 3-plasmid transient system for rAAV production of 1.6:1:1 of helper plasmid:rep/cap plasmid (i.e. packaging plasmid): transfer vector. usual. For this reason, the transfections in which BACs provided helper functions were at a molar ration of 0.62:1.0:0.86. The 3-plasmid system plasmids were used at a 1.6:1:0.86 molar ratio of helper plasmid to rep/cap plasmid to transfer vector plasmid. To one flask of cells transfected with the Cre-dependent rep/cap plasmid (pG2.AAV5.R2C5-hCG intron 3× pA HygR), 8.4 μg of pG3.CMVTO2-iCre was added. A negative control transfection containing no rep/cap plasmid (helper and transfer vector only) was also included. One flask of cells was an untransfected control. The plasmids were added to 6 ml Opti-MEM media containing 58.5 μl of PEI Pro. The transfection mixes were vortexed and incubated at room temperature for 15 minutes before being added to shaker flasks containing the cells. To one of the flasks of cells transfected with CreBAC6, DOX was added to the cell culture medium to a final concentration of 2 μg/ml. The cells were incubated at 37° C. with shaking. The following day 1 M sodium butyrate was added to each flask to a final concentration of 5 mM.

After 72 hours post-transfection, the cells were pelleted by centrifugation at 1,300 rpm for 10 minutes and resuspended in 4 ml lysis buffer. The cells were lysed by 3×cycles of freezing in dry ice plus ethanol followed by thawing at 37° C. Benzonase was then added to the lysates at 50 U/ml and the tubes incubated at 37° C. for 30 minutes. The lysate was then cleared by centrifugation at 1,300 rpm for 10 minutes after which the supernatant was aliquoted and the pellet discarded.

### AAV Transduction

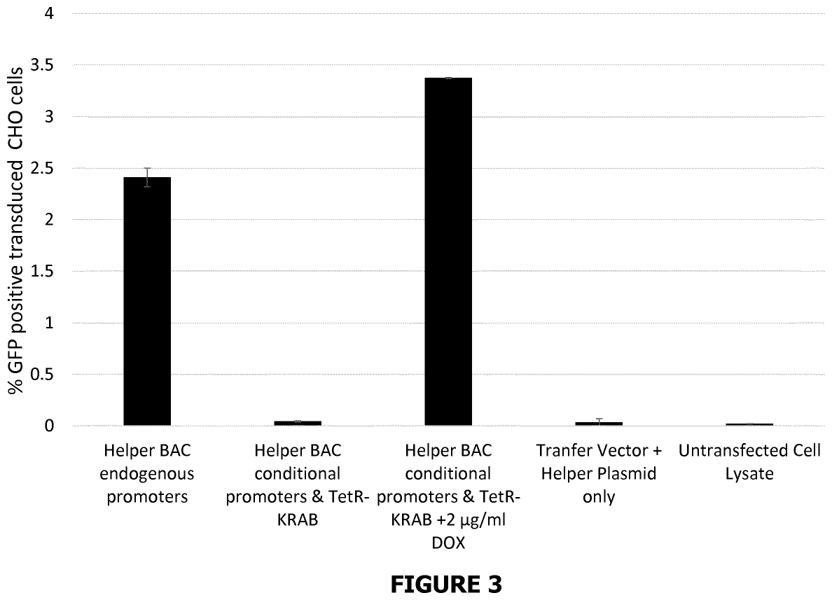

CHO cells, which are receptive to transduction with both AAV2 and AAV5, were plated in a 96 well plate at 8×10³ cells/well (growing in 200 μl per well DMEM containing 10% FCS, 1× Glutamax and 1× non-essential amino acids). The following day, 20 μl of each of the cell lysates containing the rAAV5 and the negative control lysates were added to wells of CHO cells in duplicate. The plate of CHO cells was incubated for 5 hours at 37° C. following which, the medium containing the lysates was aspirated from the wells and replaced with fresh medium. The plate was then incubated at 37° C. for a further 67 hours. The media was then aspirated from the transduced CHO cells and the cells were disaggregated with 200 μl EDTA solution and were then analysed on an Accuri C6 flow cytometer to measure the level of GFP fluorescence and also analysed with FlowJo software. The live cell population was gated on (FSC-A/SSC-A), then a gate was set up for single cells (FSC-A/FSC-H). Untransduced CHO cells were used to set the baseline fluorescence (FL1-A/FSC-A) above which cells could be considered GFP positive. The percentage of cells above the fluorescence baseline was calculated for each of the wells of transduced cells, the average and standard deviation was then calculated for each of the duplicates. The results are shown in FIG. 3.

FIG. 3 shows that BAC in which all the Ad2 helper genes retain their endogenous promoters was capable of providing helper function in transient AAV vector production. The relatively low level of transduced recipient cells (˜2.4%), reflecting a relatively low titre recombinant vector, was likely due to the sub-optimal plasmid ratio used in the transfections of the producer cells and the low transduction efficiency of such a large (>30 kb) construct into cells. In the absence of DOX in the cell culture medium, CreBAC6, in which all the helper genes are under conditional control, did not provide enough helper gene function for the transfected cells to produce enough recombinant vector to transduce CHO cells to detectable levels of fluorescence. This is due to the fact that, in the absence of DOX, cells transfected with this BAC would be constantly expressing TetR-KRAB which inhibits the transcription of all the helper genes. This shows that expression of these genes from this BAC is significantly suppressed in the absence of DOX. This is will likely reduce the level of toxicity of the Ad2 helper proteins to cells stably transfected with such a construct. Cells transfected with CreBAC6 in the presence of 2 μg/ml DOX in the cell culture medium produced recombinant AAV vector at levels high enough to transduce a greater percentage of CHO cells (˜3.4%) than those transduced with lysate from cells transfected with the helper BAC retaining the endogenous promoters. It is possible that, when relieved of TetR-KRAB repression, the CMVTO2 promoter is more active than the endogenous promoters of E2A and E4, allowing for a greater level of expression of these genes in transfected cells resulting in higher levels of recombinant vector production. This data shows that the expression of the E2A and E4 helper genes is not disrupted by replacing their native promoters with conditional promoter and may even be improved.

The cells transduced with the negative control helper plasmid+transfer vector only transfected cell lysate did not have any detectable level of fluorescence. This shows that all fluorescence measured above baseline in the transduced CHO cells was due to AAV transduction delivery of the EGFP gene into the cells and not due to EGFP protein absorption from the producer cell lysate into the cells.

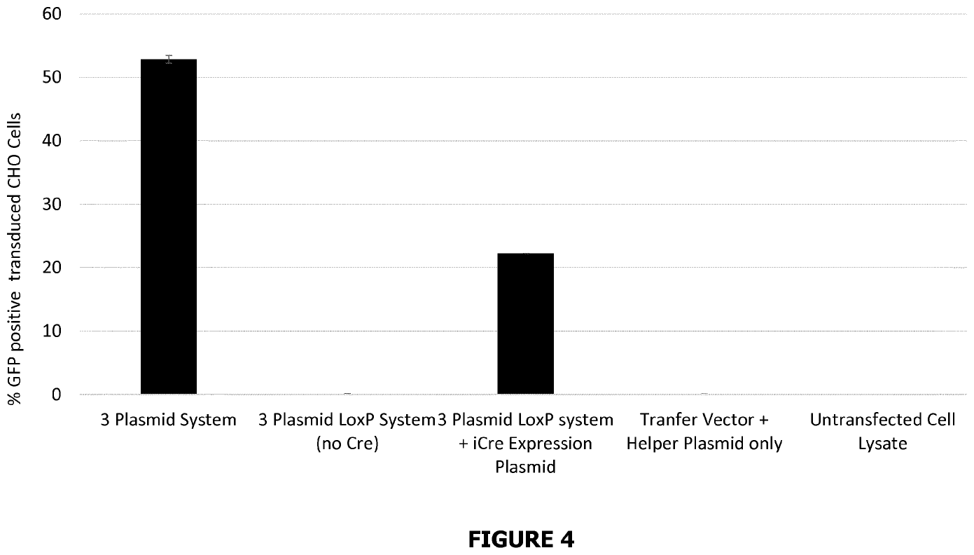

FIG. 4 shows that, as expected, cells transfected with the 3-plasmid system is capable of producing quantities of recombinant AAV5 vector enough to transduce recipient cells to high levels (˜52.3%).

Cells transfected with the Cre-dependent rep/cap expression plasmid (i.e. rep gene comprising termination intron) (pG2.AAV5.R2C5-hCG intron 3× pA HygR) along with the Ad2 helper plasmid and EGFP transfer vector in the absence of a Cre expression plasmid did not produce recombinant vector to levels capable of producing detectable fluorescence in transduced cells. This shows that, in the absence of Cre, the rep gene comprising the termination intron is functionally silent.

Cells transfected with the Cre-dependent rep/cap expression plasmid along with the Ad2 helper plasmid, EGFP transfer vector and an iCre expression plasmid (pG3.CMVTO2-iCre) produced recombinant vector enough to transduce recipient cells to high levels (˜22.2%) though not as much as cells transfected with the standard non-Cre-dependent rep/cap. This shows that this Cre-dependent rep gene (i.e. rep gene comprising termination intron) is capable of producing functional AAV vector. The lower amount of vector produced compared to the non-Cre-dependent rep/cap plasmid could be due to a number of factors other than disruption of splice variant ratios. It is possible that the delay in expression of Rep due to the requirement for iCre to first be translated and the LoxP sites to recombine, could result in lower vector yields. It is possible that the level of iCre expression in the cells was not optimal and that transfecting cells with a larger amount of the iCre expression plasmid could result in higher vector yields. It is possible that, as iCre is constantly expressed and active in the cells, the transcriptional terminators could be re-inserted into repgenes in which they have already been removed, resulting in Rep expression constantly being in flux in the cells. The Cre-dependent and standard rep/cap plasmids were purified using different kits (Qiagen Plasmid Plus Midi Kit and Nucleobond Xtra Maxi EF Kit respectively) and it could simply be the case that the quality of the standard rep/cap plasmid prep was higher.

Therefore, the data in FIG. 3 shows that replacing the endogenous promoters of the Ad2 helper genes with conditional promoters does not negatively affect their expression when they are activated. The data in FIG. 4 shows that the Cre-dependent rep is capable of producing functional recombinant AAV vector in the presence of Cre/iCre. Both the conditional helper genes and Cre-dependent rep are functionally silent when not activated. Therefore, in the stable AAV producer BAC construct as all cellular toxicity should be suppressed in cells in which the construct has integrated, allowing them to divide normally until recombinant vector production is initiated.

### Example 4

### Design of AAV Bacterial Artificial Chromosome

The method below was used to clone the elements into BAC.

1. Each element is PCR amplified using a proof reading DNA polymerase with primers that include unique restriction sites that will allow the element to be cloned into the multiple cloning site of the BAC donor plasmid, pDonor.

2. The PCR amplified fragment is gel purified and ligated into the TOPO cloning plasmid pCR-Blunt II TOPO. This ligation is used to transform chemically competent E. coli.

3. Plasmid containing the PCR amplified element is extracted from an E. coli broth culture and digested with the 2 restriction enzymes for which sites were introduced by the PCR primers.

4. The digested plasmid is separated by agarose gel electrophoresis and the digested element is excised from the gel and purified.

5. The digested and purified element is ligated into the multiple cloning site of pDonor. This plasmid contains 2 chicken hypersensitive insulator sites (2×cHS4) near the multiple cloning site. Elements are cloned into pDonor directionally so that the 2×cHS4 is situated at the 3′ end of the element. A restriction site for the meganuclease enzyme I-SceI is situated in pDonor upstream of the 5′ end of the directionally cloned element whereas a restriction site for the meganuclease enzyme PI-PspI is situated at the 3′ end of the 2×cHS4. This ligation is used to transform chemically competent E. coli.

6. The pDonor plasmid carrying the directionally cloned element is extracted from an E. coli broth culture and digested first with the meganuclease I-SceI at 37° C. and then with PI-PspI at 65° C.

7. The digested plasmid is separated by agarose gel electrophoresis and the digested element including 2×cHS4 at the 3′ end is excised from the gel and purified.

8. The BAC contains a single PI-PspI restriction site. BAC DNA is digested with PI-PspI at 65° C. and then dephosphorylated by adding with FastAP alkaline phosphatase to the digest reaction and incubating it at 37° C. for 30 minutes. The FastAP is then deactivated by incubating the reaction at 75° C. for 5 minutes.

9. The meganucleases I-SceI and PI-PspI cleave DNA leaving overhangs that are compatible, yet asymmetrical. Therefore, the I-SceI and PI-PspI digested element with 2×cHS4 at the 3′ end can be directionally ligated into the PI-PspI digested BAC. The I-SceI overhang at the 5′ end of the element will bind one of the PI-PspI overhangs in the BAC, resulting in a new sequence that can no longer be cut by either I-SceI or PI-PspI. The PI-PspI overhang at the 3′ end of the 2×cHS4 downstream of the element will bind the other PI-PspI overhang in the BAC resulting in the formation of a new PI-PspI site in the ligated BAC at the 3′ end of the 2×cHS4. This ligation is used to transform electrocompetent E. coli using an electroporator.

10. The BAC, now containing the newly cloned element with 2×cHS4 at the 3′ end, is extracted from an E. coli broth culture. This BAC can be digested with PI-PspI and dephosphorylated and further elements that have been directionally subcloned into pDonor can be ligated into this site. Any new element that has first been cloned into pDonor, when ligated into the BAC will always have 2×cHS4 at its 5′ end from the previously cloned fragment and 2×cHS4 at its 3′ end after it has been cloned into pDonor.

A schematic diagram of a nucleic acid vector produced in this way is shown in FIG. 5.

### Example 5

### Generation of Stable Pools of Cells Transfected with AAV5 BAC Constructs

Transfection and selection of the rAAV BAC constructs were performed in adherent HEK293T cells. Suspension-adapted HEK293Tsa pre-MCB cells growing in BalanCD medium in shaker culture were counted and re-suspended at 2×10⁵ cells/ml in DMEM medium containing 10% FCS, which reverts them to adherent behaviour. The cells were plated out in a 6-well plate, 2 ml per well, which was then incubated overnight at 37° C. The following day, the maxipreps of the stable AAV BAC constructs containing the EGFP transfer vector: TetR-KRAB iCreBAC9b-GFP and TetR iCreBAC9b-GFP were transfected into the plated cells. Each transfection contained 5 μg of DNA from a BAC maxiprep added to 300 μl OPTI-MEM, to which 5 μl of PEI-pro was added. The tubes were briefly vortexed and incubated for 10 minutes. Following this, the transfection mixtures were added to each well. After 48 hours, the wells were aspirated and the medium replaced with DMEM containing 10% FCS and 300 μg/ml Zeocin. The plate was incubated for several days during which most un-transfected cells died and floated off the surface of the wells. The medium was replaced several times with fresh DMEM medium containing 10% FCS and 300 μg/ml Zeocin. After 7 days the cells in the wells were mostly EGFP positive and doubling at a normal rate.

### Induction Recombinant AAV Vector Production of Stable Pools

The pools of HEK293T cells stably transfected with the TetR-KRAB iCreBAC9b-GFP and TetR iCreBAC9b-GFP constructs were counted and plated in T175 flasks at 1×10⁷ cells per flask in 25 ml DMEM containing 10% FCS and 50 μg/ml Zeocin. All the flasks of cells were incubated overnight at 37° C. The following day, vector production in the cells was induced by replacing the medium with fresh DMEM containing 10% FCS with 2 μg/ml DOX and 5 mM Sodium Butyrate. A flask of each of the two stable pools was left uninduced as a negative control. The cells of 1 induced flask and the uninduced controls were harvested 72 hours after addition of DOX. Another induced flask of each of the stable pools was harvested 96 hours after addition of DOX.

To obtain the lysate from the induced and uninduced cells, cells were pelleted by centrifugation at 1,300 rpm for 10 minutes and resuspended in 2 ml lysis buffer. The cells were then lysed by 3×cycles of freezing in dry ice plus ethanol followed by thawing at 37° C. The lysates were cleared by centrifugation at 1,300 rpm for 10 minutes after which the supernatants were removed to new tubes and the pellets discarded.

### Transduction of Recipient Cells with Lysates from Stable Producers

CHO cells, which are receptive to transduction with AAV5, were plated in a 96 well plate at 8×10³ cells/well (growing in 200 μl per well DMEM containing 10% FCS, 1× Glutamax and 1× non-essential amino acids). The following day, 20 μl of each of the cell lysates containing the rAAV5 and the negative control lysates were added to wells of CHO cells in duplicate. The plate of CHO cells was incubated for 5 hours at 37° C., following which the medium containing the lysates was aspirated from the wells and replaced with fresh medium. The plate was then incubated at 37° C. for a further 67 hours. The media was then aspirated from the transduced CHO cells and the cells were disaggregated with 200 μl EDTA solution and were then analysed on an Accuri C6 flow cytometer to measure the level of GFP fluorescence. The live cell population was gated on (FSC-A/SSC-A), then a gate was set up for single cells (FSC-A/FSC-H). Untransduced CHO cells were used to set the baseline fluorescence (FL1-A/FSC-A) above which cells could be considered GFP positive. The percentage of cells above the fluorescence baseline was calculated for each of the wells of transduced cells. None of the cells transduced with lysates from the induced stable pools showed any GFP fluorescence above baseline.

### RNAseq Analysis of Stable Pools of Cells Transfected with the AAV5 BAC Constructs

In order to determine if the constructs were mechanistically functional in the cells, RNA sequencing analysis of all polyadenylated RNA from DOX-induced and uninduced cells were compared. The pools of HEK293T cells stably transfected with the TetRKRAB iCreBAC9b-GFP and TetR iCreBAC9b-GFP constructs were counted, diluted in DMEM containing 10% FBS to 3×10⁵ cells/ml, and each plated in 6-well plates, 2 ml per well. The plates were incubated overnight at 37° C. The following day, 3 wells of each stable pool were induced by changing the medium to DMEM containing 10% FBS, 2 μg/ml DOX, and 5 mM sodium butyrate. The medium was simply replaced with fresh DMEM+10% FCS in the remaining 3 wells of each plate. The plates were incubated for 5 days at 37° C. after which the uninduced cells were harvested by disaggregating them with trypsin, resuspending them in 1 ml medium and spinning them down at 300×g to pellet the cells. The medium was aspirated and the cell pellets were stored at −80° C. The medium of the induced cells was replaced with fresh DMEM medium containing 10% FBS, 2 μg/ml DOX, and 5 mM sodium butyrate and the plates were incubated for a further 2 days at 37° C. Following this, the induced cells were also harvested, pelleted and stored at −80° C. RNA seq analysis was performed on polyadenylated RNA extracted from the cell pellets by GeneWiz using an Illumina. The reads were returned to GSK where they were aligned with the BAC sequences using IGV software (data not shown).

The alignments showed that TetR, TetR-KRAB and EGFP-fLuc, all of which are under the control of the constitutive CMV promoter in the constructs are, as expected, expressed in both DOX-induced and uninduced HEK 293T cells. However, the alignments also showed that the conditionally expressed ORFs in the constructs: E2A, E4, iCre and rep2cap5, are, as designed, transcriptionally activated in the DOX-induced cells while they could not be detected in the uninduced cells. The number of reads aligning to these ORFs in the DOX-induced cells RNA was substantially lower than the number of reads that align to the constitutively active ORFs. The fact that these transcripts could not be detected in the uninduced cells confirms that the transcription of these ORFs is blocked by TetR and by TetR-KRAB in the absence of DOX. No reads aligned to the VA ORF in the RNA from any of the cells. This was expected as only polyadenylated RNA was purified and VA, being expressed from a pol. Ill promoter, is not polyadenylated. This all confirmed that the constructs were mechanistically functional in HEK 293T cells and that the lack of detectable Rep protein or transducing vector in DOX-induced cells was most likely due to the low level of transcripts.

The number of transcripts per million (TPM) for each of the BAC construct ORFs is shown below in table 2 for both stable pools, induced and uninduced:

| | | | HEK293T | |
|---|---|---|---|---|
| | HEK293T TetR | | TetRKRAB | |
| | AAV BAC stable | | AAV BAC | |
| | pool | | stable pool | |
| BAC element | Uninduced | Induced | Uninduced | Induced |
| TetR | 3641.203 | 3813.955 | 4680.331 | 5103.172 |
| Ad2 E2A | 3.498 | 789.634 | 0.230 | 114.586 |
| Ad2 E4 | 0.072 | 3.530 | 0.016 | 19.891 |
| AAV rep2/cap5 | 20.145 | 38.072 | 17.083 | 42.005 |
| iCre | 4.869 | 220.547 | 0.519 | 81.279 |
| GFP-Luc | 6200.196 | 3526.372 | 1637.245 | 1915.951 |

The number of reads for the TetR portion of the constructs was around 3600-5100 per million transcripts. The EGFP-fLuc transcripts were present at similar levels to TetR.

Of the conditionally expressed ORFs, E2A is most highly induced by DOX in cells expressing TetR. In uninduced cells expressing TetR or TetR-KRAB no E2A transcripts could be detected. This means that the standard GSK codon-optimised TetR protein is sufficient to completely block transcription of genes downstream of the CMVTO2 promoter under normal growth conditions and that the extra heterochromatinization of the surrounding DNA by the KRAB domain in TetR-KRAB is not necessary to enhance the negative regulation in the case of this gene. Induction of E2A transcription by addition of DOX to the cell growth medium was 6.89×higher in cells stably transfected with the TetR construct than with the TetR-KRAB construct. It was possible that this was due to the increased negative regulation by the KRAB domain. The iCre gene was also expressed at higher levels (2.71×) in the induced TetR expressing cells than the induced TetR-KRAB expressing cells.

The E4 and rep2cap5 ORFs were expressed at even lower levels in both stable pools.

Inversely to E2A and iCre, the E4 ORF was expressed more highly (5.65×) in DOX induced cells expressing TetR-KRAB than those expressing TetR. One hypothesis is that E4 ORF6 is highly toxic to cells and that when the BAC constructs are first transfected into the cells, there is a window of time before the TetR or TetR-KRAB protein is expressed at sufficient levels and during which, the conditionally expressed genes downstream of the CMVTO2 promoter can be transcribed and protein produced. Due to its toxicity, it is possible that the E4 ORF is selected against and only cells in which this region of DNA has become silenced or split survive, resulting in stable cells lacking the E4 region. It is possible that cells expressing TetR-KRAB, which provides tighter negative regulation than TetR alone, are more likely to stop the E4 ORF6 protein being produced at an earlier time than cells expressing TetR, resulting in more cells surviving that have the intact E4 region of the BAC integrated into their genome. In order to avoid the E4 region of the BAC from being selected against, it may be necessary to pre-transfect the cells with a TetR/TetR-KRAB RNA so that the CMVTO2 promoter of E4 is bound and transcription blocked as soon as it enters the cells.

This also showed that rep2cap5 transcripts increase in the induced cells. The low levels of rep transcripts seen in uninduced cells were because, in HEK293 cells, the rep promoters are constitutively active. As the transcriptional terminators downstream of P19 are still in place in the absence of DOX, these transcripts represent the short, prematurely terminated RNAs. The increase in rep2cap5 transcripts in the DOX-induced stable pools is due to the removal of the transcriptional terminators, allowing transcripts to proceed through the entire ORF, although it's likely the actual number of transcripts do not actually increase. This increase in the rep2cap5 RNA in induced cells is proof that the levels of iCre in the induced cells are high enough to recombine the LoxP sites flanking the transcriptional terminators.

It was possible that within each of the stable pools, there are cells with a high number of integrations that, if cloned, would be able to produce detectable levels of recombinant AAV vector upon DOX induction.

### Construction of a Transposase Regulated Rep/cap

Additional BAC constructs were tested in which the transcriptional terminators downstream of the rep promoters are flanked by transposon ITRs and iCre is replaced with a transposase.

It has been reported that it is possible, through mutation of 3 amino acids in the cabbage looper moth (Trichoplusia ni) transposase used in the piggyBac system to create an excision+integration−phenotype (Li et al., 2013 “PiggyBac transposase tools for genome engineering” PNAS 110: E2279-E2287). This would mean that expression of the transposase by addition of DOX to cells stably transfected with such a construct would result in an irreversible removal of the transcriptional terminators downstream of the rep promoters, hopefully resulting in greater Rep expression.

The transposase from Macdunnoughia crassisigna is 98.82% identical to that from Trichoplusia ni. Yusa et al. (Yusa K et al “A hyperactive piggyBac transposase for mammalian applications, 2011, PNAS 108: 1531-1536) found 7 amino acid substitutions (I30V, S103P, G165S, M282V, S509G, N538K, N571S) in the Trichoplusia ni transposase that resulted in a hyperactive phonotype. These substitutions were applied to the M. crassisigna transposase amino acid sequence. Additionally, 3 amino acid substitutions found by Li et al. (2013, PNAS 110: E2279-E2287) to result in an excision+integration−phonotype in the Trichoplusia ni transposase (R372A, K375A, D450N) were also applied to the M. crassisigna transposase sequence. The modified M. crassisigna transposase amino acid sequence is shown below with hyperactive phenotype substitutions highlighted in red and excision+integration−phenotype substitutions highlighted in green.

The modified M. crassisigna transposase amino acid sequence (SEQ ID NO: 1) is shown below with hyperactive phenotype substitutions underlined and in bold, and excision+integration−phenotype substitutions in bold italics:

| | | | |
|---|---|---|---|
| Sbjct | 1 | MGSSINDEHILSALLQSDDELVGEDSDSEVSDHVSEDDVQSDTEEAFIDEVHEVQPTSSG | 60 |
| Sbjct | 61 | SEILDEQNVIEQPGSSLASNRTLTLPQRTIRGKNKHCWSTSKPTRRSRVSALNIVRSQRG | 120 |
| Sbjct | 121 | PTRMCRNIYDPLLCFKLFFTDEIISEIVKWTNAEISLKRRESMTSATFRDTNEDEIYAFF | 180 |
| Sbjct | 181 | GILVMTAVRKDNHMSTDDLFDRSLSMVYVSVMSRDRFDFLIRCLRMDDKSIRPTLRENDV | 240 |
| Sbjct | 241 | FTPVRKIWDLFIHQCIQNYTPGAHLTIDEQLLGFRGRCPFRVYIPNKPSKYGIKILMMCD | 300 |
| Sbjct | 301 | SGTKYMINGMPYLGRGTQTNGVPLGEYYVKELSKPVHGSCRNITCDNWFTSIPLAKNLLQ | 360 |
| Sbjct | 361 | EPYKLTIVGTV [symbol] SN [symbol] REIPEVLKNSRSRPVGTSMFCFDGPLTLVSYKPKPAKMVYLLSSC | 420 |
| Sbjct | 421 | DEDASINESTGKPQMVMYYNQTKGGVDTL [symbol] QMCSVMTCSRKTNRWPMALLYGMINIACIN | 480 |
| Sbjct | 481 | SFIIYSHNVSSKGEKVQSRKNFMRNLYMGLTSSFMRKRLEAPTLKRYLRDNISNILPKEV | 540 |
| Sbjct | 541 | PGTSDDSTEEPVTKKRTYCTYCPSKIRRKANASCKKCKKVICREHNIDMCQSCF | 594 |

This amino acid sequence was converted into a codon optimised DNA sequence and synthesised.

The ITRs from the M. crassisigna transposon (EU287451) were also synthesised.

To generate DNA fragments for the cloning of the transposon ITR flanked transcriptional terminators into the intron in rep, primers were designed (see table 3) to PCR amplify the entire sequences of GSK's in-house rep2cap5 expression plasmid pG2.AAV5.R2C5-intron a 9.15 kb fragment with the primers Int-3′ITR Gib F & Int-5′ITR Gib R. The 2.72 kb fragment containing the transcriptional terminators was PCR amplified from pUC57.Int-3A-Hyg using the primers 3×pA-5′ITR Gib F & 3×pA-3′ITR Gib R. The 349 bp 5′ transposon ITR was amplified from pUC57.5′-ITR using the primers 5′ITR-Int Gib F & 5′ITR-3×pA Gib R. The 278 bp 3′ transposon ITR was amplified from pUC57.3′-ITR using the primers 3′ITR-3×pA Gib F & 3′ITR-Int Gib R.

TABLE 3
Primer sequences

| Name | Sequence |
|---|---|
| Int-3′ITR Gib F | ATATGATTATCTTTCTAGGGTTAAATCACTGAATCCGGGAGCAC |
| (SEQ ID NO: 2) | |
| Int-5′ITR Gib R | CGCAGACTATCTTTCTAGGGTTAATTCTATGCCCAGCACG |
| (SEQ ID NO: 3) | |
| 5′ITR-Int Gib F | CGTGCTGGGCATAGAATTAACCCTAGAAAGATAGTCTGCG |
| (SEQ ID NO: 4) | |
| 5′ITR-3xpA Gib R | ATTATGATCAGAAGATCTGGGATATCTATAACAAGAAAATATATATATAATAAG |
| (SEQ ID NO: 5) | |
| 3xpA-5′ITR Gib F | ATTTTCTTGTTATAGATATCCCAGATCTTCTGATCATAATCAG |
| (SEQ ID NO: 6) | |
| 3xpA-3′ITR Gib R | ATAAAGTAACAAAACTTTTAGGATCCCGAGCTTGGCACTG |
| (SEQ ID NO: 7) | |
| 3′ITR-3xpA Gib F | CAGTGCCAAGCTCGGGATCCTAAAAGTTTTGTTACTTTATAGAAGAAATTTTGAG |
| (SEQ ID NO: 8) | |
| 3′ITR-Int Gib R | TCCCGGATTCAGTGATTTAACCCTAGAAAGATAATCATATTGTGAC |
| (SEQ ID NO: 9 | |
| PolyA-Transpose Gib | TGTGCCAATCTTGCTTCTGAGAATTCACCCCACCAGTGCAG |
| F (SEQ ID NO: 10) | |
| CMVTO2-Transpose Gib | TCGTTGATAGACGAACCCATGGTGGCGGCCTTTGCCAAAG |
| R (SEQ ID NO: 11) | |

All primers were ordered from ThermoFisher Scientific.
Underlined nucleotides denote a 5′ overhang included in the primer to provide a region of overlap in the PCR product with the sequence that it was to be assembled adjacent to in the Gibson cloning reaction.

The PCRs for the 2 transposon ITRs and the transcriptional terminators were performed followed by the amplification of the pG2.AAV5.R2C5-intron. The 2 transposon ITRs were joined either side of the 3× pA HygR transcriptional terminator fragment by overlapping PCR first. The fragments of the 5′-ITR and the transcriptional terminators were combined in a PCR using the primers 5′ITR-Int Gib F & 3×pA-3′ITR Gib R. This fragment was then combined with the 3′ transposon ITR fragment in a PCR using the primers 5′ITR-Int Gib F & 3′ITR-Int Gib R.

Equal volumes of 5 μl of the 5′ITR-3×pA-HygR-3′ITR fragment and the pG2.AAV5.R2C5-intron fragment were combined and cloned using NEBuilder HiFi DNA Assembly Mastermix.

The M. crassisigna transposase was clone downstream of the CMVTO2 promoter as follows.

To generate DNA fragments for the cloning of the M. crassisigna transposase downstream of the CMV-TO2 promoter and upstream of an SV40 polyA, primers were designed to PCR amplify pG3.CMVTO2—as a 4.25 kb fragment. The primers contained overlaps with the transposase sequence ends.

Equal volumes of 5 μl of the pG3.CMVTO2 fragment and the M. crassisigna transposase fragment were combined and cloned using NEBuilder HiFi DNA Assembly Mastermix.

### Test of the Transposase-regulated rep2cap5 to Produce Functional AAV Vector in Transiently Transfected Cells

In order to test the ability of the transposase to remove the recombinant transposon inrep and initiate AAV vector production the components were tested in transient transfection. Flasks of suspension adapted HEK 293 cells were transfected with plasmids as follow:
1. rep2/cap5+helper plasmid+EGFP transfer vector plasmid
2. rep2/cap5-transposon 3×pA+helper plasmid+EGFP transfer vector plasmid
3. rep2/cap5-transposon 3×pA+helper plasmid+EGFP transfer vector plasmid+transposase
4. rep2/cap5-LoxP 3×pA+helper plasmid+EGFP transfer vector plasmid
5. rep2/cap5-LoxP 3×pA+helper plasmid+EGFP transfer vector plasmid+iCre
6. helper plasmid+EGFP transfer vector plasmid
7. Untransfected cells

Transfection procedure was as follows.

HEK293Tsa cells were seeded in 250 ml shaker culture flasks at 2×10⁶ cells per ml, 60 ml per flask in BalanCD HEK293 media, 2% Glutamax, 0.1% Pluronic F-68. The plasmids were used at a 1.6:1:1 molar ratio of helper plasmid to rep/cap plasmid to transfer vector plasmid. The plasmids were added to 6 ml Opti-MEM media containing 58.5 μl of PEI Pro. The transfection mixes were vortexed and incubated at room temperature for 15 minutes before being added to shaker flasks containing the cells. The cells were incubated at 37° C. with shaking. The following day, 1 M sodium butyrate was added to each flask to a final concentration of 5 mM.

After 72 hours post-transfection, the cells were pelleted by centrifugation at 1,300 rpm for 10 minutes and resuspended in 4 ml lysis buffer. The cells were lysed by 3× cycles of freezing in dry ice plus ethanol followed by thawing at 37° C. Benzonase was then added to the lysates at 50 U/ml and the tubes incubated at 37° C. for 30 minutes. The lysate was then cleared by centrifugation at 1,300 rpm for 10 minutes after which the supernatant was harvested and the pellet discarded.

CHO cells, which are receptive to transduction with AAV5, were plated in a 96 well plate at 8×10³ cells/well (growing in 200 μl per well DMEM containing 10% FCS, 1× Glutamax and 1× non-essential amino acids). The following day, 20 μl of each of the cell lysates containing the rAAV5 and the negative control lysates were added to wells of CHO cells in duplicate. The plate of CHO cells was incubated for 5 hours at 37° C. following which, the medium containing the lysates was aspirated from the wells and replaced with fresh medium. The plate was then incubated at 37° C. for a further 67 hours. The media was then aspirated from the transduced CHO cells and the cells were disaggregated with 200 μl EDTA solution and were then analysed on a flow cytometer to measure the level of GFP fluorescence. The live cell population was gated on (FSC-A/SSC-A), then a gate was set up for single cells (FSC-A/FSC-H). Untransduced CHO cells were used to set the baseline fluorescence (FL1-A/FSC-A) above which cells could be considered GFP positive. The percentage of cells above the fluorescence baseline was calculated for each of the wells of transduced cells, the average and standard deviation was then calculated for each of the duplicates. These are shown in table 4:

TABLE 4
percentage of cells above the fluorescence baseline

| | |
|---|---|
| Ad2 Helper + rep2cap5 + EGFP transfer | 42.05 |
| Ad2 Helper + rep2cap5-transposon + EGFP | 0.535 |
| transfer | |
| Ad2 Helper + rep2cap5-transposon + EGFP | 20.55 |
| transfer + transposase | |
| Ad2 Helper + rep2cap5-loxP + EGFP transfer | 0.0235 |
| Ad2 Helper + rep2cap5-loxP + EGFP transfer + | 22.4 |
| iCre | |
| Ad2 Helper + EGFP Transfer | 0.0395 |
| Untransfected | 0 |

### Comparison of Transduction with rAAV Produced Using Constitutive, Transposase-dependent, and Cre-dependent rep2/cap5

This data shows that, as expected, cells transfected with the standard 3-plasmid system are capable of producing quantities of recombinant AAV5 vector, enough to transduce recipient cells to high levels (˜42%).

Cells transfected with the transposon-dependent rep/cap expression plasmid (pG2.AAV5.R2C5-intron transposable 3× pA) along with the Ad2 helper plasmid and EGFP transfer vector in the absence of a transposase expression plasmid did not produce recombinant vector to levels capable of producing detectable fluorescence in transduced cells. This shows that, in the absence of transposase, this recombinant rep/cap is functionally silent and due to the transcriptional terminators.

Cells transfected with the transposon-dependent rep/cap expression plasmid along with the Ad2 helper plasmid, EGFP transfer vector and the M. crassisigna transposase expression plasmid (pG3.CMVTO2-M. crassisigna transposase) produced enough recombinant vector to transduce recipient cells to high levels (˜20.6%). This was comparable to the amount of recombinant vector produced when the Cre-dependent rep/cap expression plasmid (pG2.AAV5.R2C5-hCG intron 3× pA HygR) was co-transfected with Ad2 helper plasmid, EGFP transfer vector and the iCre expression plasmid (pG3.CMVTO2-iCre) (22.4%), though not as much as cells transfected with the standard non-transposase-dependent rep/cap. This indicates that this transposase-dependent rep gene is capable of producing functional AAV vector. The lower amount of vector produced compared to the non-transposase-dependent rep/cap plasmid could be due to a number of factors. It is possible that the delay in expression of Rep due to the requirement for transposase to first be translated and the ITRs to recombine, could result in lower vector yields. It is possible that the level of transposase expression in the cells was not optimal and that transfecting cells with a greater amount of the transposase expression plasmid could result in higher vector yields.

This data shows that the transposase-dependent rep is capable of producing functional recombinant AAV vector in the presence of transposase. The transposase-dependent rep is functionally silent when not activated. Unlike the Cre-dependent rep gene, removal of the transcriptional terminators with the excision positive/integration negative recombinant transposase is not reversable. This should result in a more stable expression of Rep protein following induction of stable cells.

1. An adeno-associated virus (AAV) vector producer cell comprising nucleic acid sequences encoding:
AAV rep and cap genes,
helper virus genes, and
a DNA genome of the AAV vector;
wherein the AAV rep gene comprises an intron, said intron comprising a transcription termination sequence with a first recombination site located upstream and a second recombination site located downstream of said transcription termination sequence; and
wherein said nucleic acid sequences are all integrated together at a single locus within the AAV vector producer cell genome.

2. (canceled)

3. The AAV vector producer cell according to claim 1, further comprising nucleic acid sequence encoding a recombinase gene under control of an inducible promoter and/or a steroid hormone receptor ligand-binding domain operably linked to the recombinase.

4. The AAV vector producer cell according to claim 3, wherein said nucleic acid sequences encoding the recombinase gene are integrated together with the nucleic acid sequences encoding the AAV rep and cap genes, the helper virus genes and the DNA genome of the AAV vector at a single locus within the AAV vector producer cell genome.

5.-6. (canceled)

7. The AAV vector producer cell according to claim 1, wherein the steroid hormone receptor ligand-binding domain is an estrogen receptor ligand binding-domain (ER).

8. The AAV vector producer cell according to claim 3, wherein the recombination site is a LoxP site and the recombinase gene is a cre recombinase gene, or the recombination site is a transposon ITR and the recombinase gene is a transposase gene.

9.-11. (canceled)

12. The AAV vector producer cell according to claim 1, wherein one or more of the helper virus genes are under transcriptional control, wherein the transcriptional control comprises a pCMV-TO2 promoter.

13. The AAV vector producer cell according to claim 3, wherein the inducible promoter comprises a pCMV-TO2 promoter.

14. The AAV vector producer cell according to claim 1, further comprising an insulator, present between each of the nucleic acid sequences.

15. (canceled)

16. A nucleic acid vector comprising a non-mammalian origin of replication and the ability to hold at least 25 kilobases (kb) of DNA, characterized in that said nucleic acid vector comprises nucleic acid sequences encoding:
AAV rep and cap genes;
helper virus genes; and
a DNA genome of an AAV vector;
wherein the rep gene comprises an intron, said intron comprising a transcription termination sequence with a first recombination site located upstream and a second recombination site located downstream of the transcription termination sequence; and
wherein the nucleic acid sequences encoding the AAV rep and cap genes, each of the helper virus genes and the DNA genome of the AAV vector are arranged as individual expression cassettes within the nucleic acid vector.

17. (canceled)

18. The nucleic acid vector of claim 16, further comprising nucleic acid sequences encoding a recombinase gene, arranged as an individual expression cassette within the nucleic acid vector.

19. The nucleic acid vector according to claim 18, further comprising a recombinase control element, wherein the recombinase control element comprises an inducible promoter operably linked to a recombinase gene and/or a steroid hormone receptor ligand-binding domain fused to the recombinase gene.

20. (canceled)

21. The nucleic acid vector according to claim 19, wherein the steroid hormone receptor ligand-binding domain is an estrogen receptor ligand binding-domain (ER).

22. The nucleic acid vector according to claim 21, wherein the ER is operably linked upstream and downstream of the recombinase gene.

23. The nucleic acid vector according to claim 18, wherein the recombination site is a LoxP site and the recombinase gene is a cre recombinase gene, or the recombination site is a transposon ITR and the recombinase gene is a transposase gene.

24.-26. (canceled)

27. The nucleic acid vector according to claim 16, wherein one or more of the helper virus genes are under transcriptional control as an individual expression cassette.

28. The nucleic acid vector according to claim 16, further comprising an insulator present between each of the expression cassettes.

29. (canceled)

30. The nucleic acid vector according to claim 16, wherein the vector is selected from one of: a bacterial artificial chromosome, a yeast artificial chromosome, a P1-derived artificial chromosome, a fosmid or a cosmid.

31. A method of producing a stable AAV vector producer cell line, comprising:
(a) introducing the nucleic acid vector of according to claim 16 into a culture of mammalian host cells; and
(b) selecting within the culture for a mammalian host cell which has the nucleic acid sequences encoded on the vector integrated into an endogenous chromosome of the mammalian host cell.

32. An AAV vector producer cell obtained by the method of claim 31.

33. A method of producing a replication defective AAV vector, comprising:
(a) introducing the nucleic acid vector of according to claim 16 into a culture of mammalian host cells; and
(b) selecting within the culture for a mammalian host cell which has the nucleic acid sequences encoded on the vector integrated into an endogenous chromosome of the mammalian host cell; and
(c) further culturing the selected mammalian host cell under conditions in which the replication defective AAV vector is produced.

34. A replication defective AAV vector obtained by the method of claim 33.

35. (canceled)
